(19) United States
(12) Patent Application Publication
(10) Pub. No.: US 2009/0054491 A1
(43) Pub. Date: Feb. 26, 2009

(54) Use

(75) Inventors: Louise Edwards, Mississauga (CA); Methvin Isaac, Brampton (CA); Abdelmalik Slassi, Mississauga (CA); Mats Nagard, Molndal (SE); Leonard Storlien, Molndal (SE); David Morgan, Cheshire (GB)

Correspondence Address: BIRCH, STEWART, KOLASCH & BIRCH, LLP, P.O. BOX 747, FALLS CHURCH, VA, 22040-0747, US

(73) Assignee: AstraZeneca AB, Södertälje (SE)

(21) Appl. No.: 11/921,951

(22) PCT Filed: Jun. 19, 2006
(86) PCT No.: PCT/US2006/023791

§ 371 Date: Dec. 11, 2007

Related U.S. Application Data
(60) Provisional application No. 60/694,242, filed on Jun. 28, 2005.

(51) Int. Cl. A61K 31/4439; A61P 3/04

(52) U.S. Cl. 514/340

(57) ABSTRACT

The present invention relates to the use of compounds of formula II for the treatment or prevention of obesity.

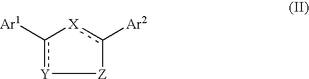

### FIELD OF THE INVENTION

The present invention relates to the use of certain compounds for the treatment or prevention of obesity.

### BACKGROUND OF THE INVENTION

The compounds of formula II below have been described in WO01/12627 A1 and WO02/068417 A2 as mGluR5 antagonists. The compounds have therein been described as being useful in the treatment of various CNS disorders such as senile dementia, schizophrenia, Alzheimer's disease and anxiety.

The metabotropic glutamate receptors (mGluR) are G-protein coupled receptors that are involved in the regulation and activity of many synapses in the central nervous system (CNS). Eight metabotropic glutamate receptor subtypes have been identified and are subdivided into three groups based on sequence similarity. Group I consists of mGluR1 and mGluR5. These receptors activate phospholipase C and increase neuronal excitability. Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002), 538.3, pp. 773-786; Pin, J-P et al., European Journal of Pharmacology (1999), 375, pp. 277-294; Bräuner-Osborne, H et al. Journal of Medicinal Chemistry (2000), 43, pp. 2609-2645; Schoepp, D. D, Jane D. E. Monn J. A. Neuropharmacology (1999), 38, pp. 1431-1476).

The involvement of the metabotropic glutamate receptor subtype 5 in central reward pathways has recently been suggested (Bradbury M. J. et al. The Journal of Pharmacology and Experimental Therapeutics, 313 (2005), 395-402).

The prevalence of obesity is increasing. At present, more than half of the U.S. population is overweight. Obesity increases a person's risk of illness and death due to diabetes, stroke, coronary artery disease, high cholesterol, hypertension as well as kidney and gall bladder disorders. Furthermore, obesity may increase the risk for some types of cancer. It is also a risk factor for the development of osteoarthritis and sleep apnea.

The object of the present invention was to find a new way for the treatment or prevention of obesity.

### OUTLINE OF THE INVENTION

The present invention is directed to the use of a compound of formula II

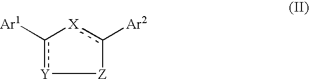

wherein X, Y, and Z are independently selected from the group consisting of N, O, S, C, and CO wherein at least one of X, Y, and Z is a heteroatom;
Ar¹ and Ar² are independently selected from the group consisting of a heterocyclic or fused heterocyclic moiety containing 1 to 4 heteroatoms selected from the group consisting of N, O, and S and an aromatic moiety selected from the group consisting of phenyl, benzyl, 1-naphthyl, 2-naphthyl, fluorenyl, anthrenyl, indenyl, phenanthrenyl, and benzonaphthenyl, wherein the Ar¹ and Ar² moieties are optionally substituted with one or more moieties selected from the group consisting of —F, —Cl, —Br, —I, —OR, —SR, —SOR, —SO₂R, —SO₂NRR′, —OCOR, —OCONRR′, —NRCOR′, —NRCO₂R′, —CN, —NO₂, —CO₂R, —CONRR′, —C(O)R, —CH(OR)R′, —CH₂(OR), —R, and -A-(CH₂)n—NRR′;
wherein R or R′ is selected from the group consisting of H, CF₃, C₁-C₁₀ alkyl, cycloalkyl, allyl-aryl, alkyl-heteroaryl, heterocycloalkyl, aryl and where R and R′ may combine to form a ring, and A is defined as CH₂, O, NH, S, SO, SO₂ and n is 1, 2, 3, or 4; and
wherein the heterocyclic or fused heterocylic moiety preferably is selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl;
or a pharmaceutically acceptable salt or an optical isomer thereof, for the manufacture of a medicament for the treatment or prevention of obesity.

The Ar¹ moiety is generally defined as a heterocyclic moiety, and the Ar¹ moiety is generally defined as a carbocylic moiety. Ar¹ and Ar² can be monocyclic or fused bicyclic groups. In one embodiment, Ar² is defined as an aryl or alkaryl moiety. In one embodiment, Ar¹ is defined as a heterocyclic, heteroaryl or heteroarylalkyl moiety. The ring systems encompassed by Ar¹ can contain up to four heteroatoms, independently selected from the group consisting of N, S, and O. When Ar¹ is a heteroaryl ring or ring system, it preferably contains one or two heteroatoms. At least one of the heteroatoms preferably is nitrogen (N). The heterocyclic or fused heterocylic moiety preferably is selected from the group consisting of quinolyl, quinazolyl, quinoxalyl, 2-pyrimidyl, 4-pyrimidyl, 5-pyrimidyl, 2-pyridyl, 3-pyridyl, 4-pyridyl, and pyrazyl.

Monocyclic Ar¹ groups include, but are not limited to: thiazoyl, furyl, pyranyl, 2H-pyrrolyl, thienyl, pyrroyl, imidazoyl, pyrazoyl, pyridyl, pyrazinyl, pyrimidinyl, and pyridazinyl moieties. Monocyclic Ar² group include but are not limited to phenyl and benzyl. Fused bicyclic Ar² include, but are not limited to, naphthyl, fluorenyl, anthrenyl, indenyl, phenanthrenyl, and benzonaphthenyl. Fused bicyclic Ar¹ groups include, but are not limited to: benzothiazole, benzimidazole, 3H-indolyl, indolyl, indazoyl, purinyl, quinolizinyl, isoquinolyl, quinolyl, phthalizinyl, naphthyridinyl, quinazolinyl, cinnolinyl, isothiazolyl, quinoxalinyl indolizinyl, isoindolyl, benzothienyl, benzofuranyl, isobenzofuranyl, and chromenyl moieties. In one embodiment, Ar¹ is a 2-pyridyl moiety. In one embodiment, Ar² is a substituted phenyl moiety.

The Ar¹ and Ar² moieties optionally may independently be substituted with one or more moieties selected from the group consisting of halogen, C₁-C₃ alkyl, C₁-C₃ O-alkyl, —OH, —OCF₃, —COOR, —COR, —SOR, —SO₂NRR′, —NRR′, —CN, —CF₃, —CO—NRR′, -A-(CH₂)ₙ—NRR′, wherein A is C, O, N, SO, SO₂, and R and R′ are independently selected from the group consisting of C₁-C₃ alkyl, H, cycloalkyl, heterocycloalkyl, aryl, and n is 1, 2, 3, or 4.

In one embodiment of the invention, the compound is selected from the group consisting of 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(3-methoxyphenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 2-(3-chlorophenyl)-4-(pyridin-2-yl)-1,3-oxazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole and pharmaceutically acceptable salts thereof.

In a further embodiment of the invention, the compound is selected from the group consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methylphenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2 pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-(3-chlorophenyl)-4-(pyridin-2-yl)-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 2-(3-bromophenyl)-4-(pyridin-2-yl)-1,3-oxazole, 2-(3-cyanophenyl)-4-(pyridin-2-yl)-1,3-oxazole, 5-(3-hydroxyphenyl)-3-(pyridine-2-yl)-1,2-oxazole, 5-(3-cyanophenyl)-3-(pyridin-2-yl)-1,2-oxazole, 3-(2-pyridyl)-5-(3-iodophenyl)-1,2,4-triazole, 3-(2-pyridyl)-5-(5-chloro-2-aminophenyl)-1,2,4-oxadiazole and pharmaceutically acceptable salts thereof.

In a further embodiment of the invention, the compound is selected from the group consisting of 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4 oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole, 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methyl phenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3 oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-(3-chlorophenyl)-4-(pyridin-2-yl)-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole and pharmaceutically acceptable salts thereof.

In still a further aspect of the invention, the compound is selected from the group consisting of 3-(5-Methyl-pyrid-2-yl)-5-(3-cyanophenyl)-1,2,4-oxadiazole, 3-(5-Cyano-pyrid-2-yl)-5-(3-cyanophenyl)-1,2,4-oxadiazole, 3-(2-Pyridyl)-5-(5-bromo-2-methoxyphenyl)-1,2,4-oxadiazole, 3-(2-Pyridyl)-5-(5-bromo-2-fluorophenyl)-1,2,4-oxadiazole, 3-(2-Pyridyl)-5-(5-cyano-2-fluorophenyl)-1,2,4-oxadiazole, 3-(2-Pyridyl)-5-(5-bromopyrid-3-yl)-1,2,4-oxadiazole, 3-(2-Pyridyl)-5-(5-chloro-pyrid-3-yl)-1,2,4-oxadiazole, 3-(5-Cyanopyrid-2-yl)-5-(5-bromo-pyrid-3-yl)-1,2,4-oxadiazole, 3-(5-Fluoropyrid-2-yl)-5-(5-bromo-pyrid-3-yl)-1,2,4-oxadiazole, 3-(2-Pyridyl)-5-(2-thiomethoxy-pyrid-3-yl)-1,2,4-oxadiazole, 3-(2-Pyridyl)-5-(5-methylpyrid-3-yl)-1,2,4-oxadiazole, 3-(2-Pyridyl)-5-(5-methoxypyrid-3-yl)-1,2,4-oxadiazole, 3-(2-Pyridyl)-5-(3-cyano-5-methylphenyl)-1,2,4-oxadiazole, 3-(2-Pyridyl)-5-(3-fluoro-5-bromophenyl)-1,2,4-oxadiazole, 3-(2-Pyridyl)-5-(3-iodo-5-bromophenyl)-1,2,4-oxadiazole, 3-(5-Fluoro-2-pyridyl)-5-(3-fluoro-5-bromophenyl)-1,2,4-oxadiazole, 3-(2-Pyridyl)-5-(3-iodo-5-(methylphenylester)-1,2,4-oxadiazole, 3-(2-Pyridyl)-5-(3-methoxy-5-(methoxycarbonyl)phenyl)-1,2,4-oxadiazole, 3-(2-Pyridyl)-5-(3-bromo-5-cyanophenyl)-1,2,4-oxadiazole, 3-(2-Pyridyl)-5-(5-cyano-3-iodophenyl)-1,2,4-oxadiazole, 3-(5-Cyano-2-pyridyl)-5-(3-bromophenyl)-1,2,4-oxadiazole, 3-(5-Cyano-2-pyridyl)-5-(3-cyano-5-fluorophenyl)-1,2,4-oxadiazole, 3-(5-Cyano-2-pyridyl)-5-(3-bromo-5-fluorophenyl)-1,2,4-oxadiazole, 3-(2-Pyridyl)-5-(5-cyano-2-methoxyphenyl)-1,2,4-oxadiazole, 3-(2-Pyridyl)-5-(2-cyano-5-methoxyphenyl)-1,2,4-oxadiazole, 3-(2-Pyridyl)-5-(5-cyano-pyrid-3-yl)-1,2,4-oxadiazole, 3-(2-Pyridyl)-5-(3-cyano-5-(methoxycarbonyl)phenyl)-1,2,4-oxadiazole, 3-(2-Pyridyl)-5-(5-phenyl-pyrid-3-yl)-1,2,4-oxadiazole, 3-(2-Pyridyl)-5-(3-cyano-5-methoxyphenyl)-1,2,4-oxadiazole, 3-(2-Pyridyl)-5-(3-cyano-5-hydroxyphenyl)-1,2,4-oxadiazole, 3-(2-Pyridyl)-5-(3-cyano-5-propoxyphenyl)-1,2,4-oxadiazole, 2-(3-Cyanophenyl)-4-(pyridin-2-yl)-1,3-thiazole, 2-((3-Bromo-5-iodophenyl)-4-pyridin-2-yl)-1,3-oxazole, 2-(2-Pyridyl)-5-(3-iodophenyl)-1,3,4-oxadiazole, 2-(2-Pyridyl)-5-(3-cyanophenyl)-1,3,4-oxadiazole, 2-(2-Pyridyl)-5-(3-cyanophenyl)-1,3,4-triazole, 3-(5-Chloropyrid-2-yl)-5-(3-cyano-5-fluorophenyl)-1,2,4-oxadiazole, 3-(5-Chloropyrid-2-yl)-5-(3-cyano-5-chlorophenyl)-1,2,4-oxadiazole, 3-(5-Chloropyrid-2-yl)-5-(3-chloro-5-fluorophenyl)-1,2,4-oxadiazole, 3-(5-Chloropyrid-2-yl)-5-(3-cyano-5-methoxyphenyl)-1,2,4-oxadiazole, 3-(5-Fluoropyrid-2-yl)-5-(3-cyano-5-chlorophenyl)-1,2,4-oxadiazole, 3-(5-Fluoropyrid-2-yl)-5-(3-fluoro-5-chlorophenyl)-1,2,4-oxadiazole, 3-(5-Fluoropyrid-2-yl)-5-(3-cyano-5-methoxyphenyl)-1,2,4-oxadiazole, 3-(5-Cyanopyrid-2-yl)-5-(3-cyano-5-chlorophenyl)-1,2,4-oxadiazole, 3-(5-Cyanopyrid-2-yl)-5-(3-fluoro-5-chlorophenyl)-1,2,4-oxadiazole, 3-(5-Cyanopyrid-2-yl)-5-(3-cyano-5-methoxyphenyl)-1,2,4-oxadiazole, 3-(5-Fluoropyrid-2-yl)-5-(3,5-di-cyanophenyl)-1,2,4-oxadiazole, 3-(3-(4-Dimethylaminobutoxy)-pyrid-2-yl)-5-(3-cyano-5-fluorophenyl)-1,2,4-oxadiazole, 3-(3-(5-Dimethylaminopentyloxy)-pyrid-2-yl)-5-(3-Cyano-5-fluorophenyl)-1,2,4-oxadiazole, 3-(3-(6-Dimethylaminohexyloxy)-pyrid-2-yl)-5-(3-cyano-5-fluorophenyl)-1,2,4-oxadiazole and pharmaceutically acceptable salts thereof.

In still a further aspect of the invention, the compound is selected from the group consisting of 3-(2-Pyridyl)-5-(3-allyloxy-5-(methoxycarbonyl)phenyl)-1,2,4-oxadiazole, 3-(2-Pyridyl)-5-(3-N,N-dimethylaminophenyl)-1,2,4-oxadiazole, 3-(2-Pyridyl)-5-(3-cyano-5-(4-pyridyl)phenyl)-1,2,4-oxadiazole, 3-(2-Pyridyl)-5-(2-methoxy-5-(4-pyridyl)phenyl-1,2,4-oxadiazole, 3-(2-pyridyl)-5-(2-fluoro-5-(4-pyridyl)phenyl)-1,2,4-oxadiazole, 3-(2-Pyridyl)-5-(3-fluoro-5-(4-pyridyl)phenyl)-1,2,4-oxadiazole, 3-(2-Pyridyl)-5-(3-fluoro-5-(3-pyridyl)phenyl)-1,2,4-oxadiazole, 3-(2-Pyridyl)-5-(2-fluoro-5-(3-pyridyl)phenyl)-1,2,4-oxediazole, 3-(2-Pyridyl)-5-(2-methoxy-5-(3-pyridyl)phenyl)-1,2,4-oxadiazole, 3-(2-Pyridyl)-5-(3-cyano-5-(3-pyridyl)phenyl)-1,2,4-oxadiazole, 3-(5-Fluoro-2-pyridyl)-5-(3-fluoro-5-(3-pyridyl)phenyl)-1,2,4-oxadiazole, 3-(2-Pyridyl)-5-(5-(3-pyridyl-pyrid-3-yl))-1,2,4-oxadiazole, 3-(5-Fluoropyrid-2-yl)]-5-[5-(3-pyridyl-pyrid-3-yl)]-3-1,2,4-oxadiazole, 3-(5-Cyanopyrid-2-yl)-5-(3-(pyrid-3-yl)phenyl)-1,2,4-oxadiazole, 3-(5-Cyanopyrid-2-yl)-5-(3-fluoro-5-(pyrid-3-yl)phenyl)-1,2,4-oxadiazole, 3-(2-Pyridyl)-5-(3-cyano-5-(2-pyridyl)phenyl)-1,2,4-oxadiazole, 3-(2-Pyridyl)-5-(2-methoxy-5-(2-pyridyl)phenyl)-1,2,4-oxediazole, 3-(2-Pyridyl)-5-(2-fluoro-5-(2-pyridyl)phenyl)-1,2,4-oxadiazole, 3-(2-Pyridyl)-5-((3-(3-fluorophenyl)-5-fluorophenyl))-1,2,4-oxadiazole, 3-(2-Pyridyl)-5-(3-cyano-5-(3-thiophene)phenyl)-1,2,4-oxadiazole, 3-(2-Pyridyl)-5-(5-(3-thienyl)-pyrid-3-yl)-1,2,4 oxadiazole, 3-(2-Pyridyl)-5-(5-3-furyl)-pyrid-3-yl)-1,2,4-oxadiazole, 3-(2-Pyridyl)-5-(5-(3-methoxyphenyl)-pyrid-3-yl)-1,2,4-oxadiazole, 3-(2-Pyridyl)-5-(3-cyano-5-(5-pyrimidyl)phenyl)-1,2,4-oxadiazole, 3-(2-Pyridyl)-5-(3-cyano-5-(3-aminophenyl)phenyl)-1,2,4-oxadiazole, 3-(2-Pyridyl)-5-(3-cyano-5-(3-fluorophenyl)phenyl)-1,2,4-oxediazole, 3-(2-Pyridyl)-5-(5-(5-pyrimidyl)-pyrid-3-yl)-1,2,4-oxadiazole, 3-(2-Pyridyl)-5-(3-aminomethyl-5-cyanophenyl)-1,2,4-oxadiazole, 3-(2-Pyridyl)-5-(5-(2-propenyl)-pyrid-3-yl)-1,2,4-oxadiazole, 3-(2-Pyridyl)-5-(3-cyano-5-vinylphenyl)-1,2,4-oxadiazole, 3-(2-Pyridyl)-5-(3-cyano-5-(2-hydroxyethyl)phenyl)-1,2,4-oxadiazole, 3-(2-Pyridyl)-5-(3-cyano-5-(2,3-dichloropropoxy)phenyl)-1,2,4-oxadiazole, 3-(2-Pyridyl)-5-(3-allyloxy-5-carboxyphenyl)-1,2,4-oxadiazole, 3-(2-Pyridyl)-5-(3-allyloxy-5-cyanophenyl)-1,2,4-oxadiazole, 3-(2-Pyridyl)-5-(5-cyano-3-(3-hydroxypropyn-1-yl)phenyl)-1,2,4-oxadiazole, 3-(2-Pyridyl)-5-(2-N-methylaminophenyl)-1,2,4-oxadiazole, and 3-(2-Pyridyl)-5-(5-(3-N-benzyl-1,2,5,6-tetrahydropyridine)-pyrid-3-yl)-1,2,4-oxadiazole.

In still a further aspect of the invention, the compound is selected from the group consisting of 3-(2-pyridyl)-5-(3-fluorophenyl)-1,2,4-oxadiazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 3-(2-pyridyl)-5-(5-chloro-2-aminophenyl)-1,2,4-oxadiazole, 5-(3-hydroxyphenyl)-3-(pyridin-2-yl)-1,2-oxazole, 5-(3-cyanophenyl)-3-(pyridin-2-yl)-1,2-oxazole, 3-(2-pyridyl)-5-(3-chlorophenyl)-1,2,4-triazole, 3-(2-pyridyl)-5-(3-iodophenyl)-1,2,4-triazole, 3-(2-pyridyl)-5-(5-hydroxypyrid-3-yl)-1,2,4-oxadiazole, 3-(5-fluoropyrid-2-yl)-5-(5-fluoro-3-(thiomethyl)phenyl)-1,2,4-oxadiazole, 5-(2-pyridyl)-3-(3-(1H-imidazol-1-yl)-5-fluorophenyl))-1,2,4-oxadiazole, 3-(2-pyridyl)-5-(3-cyano-5-trifluoromethylphenyl)-1,2,4-oxadiazole, 3-(5-fluoro-2-pyridyl)-5-(3-fluoro-5-trifluoromethylphenyl)-1,2,4-oxadiazole, 3-(5-cyanopyrid-2-yl)-5-(3-alloxy-5-cyanophenyl)-1,2,4-oxadiazole, 3-(5-fluoropyrid-2-yl)-5-(3-alloxy-5-cyanophenyl)-1,2,4-oxadiazole, 3-(5-cyanopyrid-2-yl)-5-(3-cyano-5-propoxyphenyl)-1,2,4-oxadiazole, 3-(5-fluoropyrid-2-yl)-5-(3-cyano-5-propoxyphenyl)-1,2,4-oxadiazole, 3-(2-pyridyl)-5-(3-cyano-5-nitrophenyl)-1,2,4-oxadiazole, 3-(5-fluoropyrid-2-yl)-5-(3-cyano-5-nitrophenyl)-1,2,4-oxadiazole, 3-(5-fluoropyrid-2-yl)-5-(3-cyano-5-dimethylaminophenyl)-1,2,4-oxadiazole, 3-(5-fluoropyrid-2-yl)-5-(3-cyano-5-(2-methoxyethoxy)phenyl)-1,2,4-oxadiazole, 3-(5-fluoropyrid-2-yl)-5-(3-cyano-5-(1H-imidazol-1-ylmethyl)phenyl)-1,2,4-oxadiazole, 3-(2-pyridyl)-5-(3-cyano-5-(methoxymethyl)phenyl)-1,2,4-oxadiazole, 3-(3-cyano-5-methoxyphenyl)-5-(2-pyridyl)-1,2,4-oxadiazole, 3-(3-cyano-5-methoxyphenyl)-5-(5-fluoropyrid-2-yl)-1,2,4-oxadiazole, 3-(5-fluoropyrid-2-yl)-5-(3-cyano-5-ethoxyphenyl)-1,2,4-oxadiazole, 3-(5-cyanopyrid-2-yl)-5-(3-cyano-5-ethoxyphenyl)-1,2,4-oxadiazole, 3-(5-chloropyrid-2-yl)-5-(3-allyloxy-5-cyanophenyl)-1,2,4-oxadiazole, 3-(5-chloropyrid-2-yl)-5-(3-cyano-5-propoxyphenyl)-1,2,4-oxadiazole, 3-(5-chloropyrid-2-yl)-5-(3-cyano-5-ethoxyphenyl)-1,2,4-oxadiazole, 3-(5-fluoropyrid-2-yl)-5-(3-cyano-5-hexyloxyphenyl)-1,2,4-oxadiazole, 3-(5-fluoropyrid-2-yl)-5-(3-cyano-5-(methoxymethyl)phenyl)-1,2,4-oxadiazole, 3-(5-fluoropyrid-2-yl)-5-(5-cyano-2-methoxyphenyl)-1,2,4-oxadiazole, 5-(5-cyano-2-methoxyphenyl)-3-(5-cyano-pyrid-2-yl)-1,2,4-oxadiazole, 3-(5-fluoro-pyrid-2-yl)-5-(3-bromophenyl)-1,2,4-oxadiazole, 5-(3-chloro-5-methyl-pyrid-4-yl)-3-(2-pyridyl)-1,2,4-oxadiazole, 5-(3-chloro-5-methoxy-pyrid-4-yl)-3-(2-pyridyl)-1,2,4-oxadiazole, 3-(3-cyano-5-methylphenyl)-5-(2-pyridyl)-1,2,4-oxadiazole, 3-(5-bromo-pyrid-3-yl)-5-(2-pyridyl)-1,2,4-oxadiazole, 3-(3-cyano-5-fluorophenyl)-5-(2-pyridyl)-1,2,4-oxadiazole, 3-(3-iodophenyl)-5-(2-pyridyl)-1,2,4-oxadiazole, 3-(3-cyanophenyl)-5-(2-pyridyl)-1,2,4-oxadiazole, 3-(3-cyano-5-dimethylamino-phenyl)-5-(2-pyridyl)-1,2,4-oxadiazole, 3-(3-cyano-5-methylphenyl)-5-(5-fluoro-pyrid-2-yl)-1,2,4-oxadiazole, 3-(3-cyano-5-fluorophenyl)-5-(5-fluoro-pyrid-2-yl)-1,2,4-oxadiazole, 3-(4-cyanophenyl)-3-(6-cyano-pyrid-2-yl)-1,2,4-oxadiazole, 5-(3-cyano-5-trifluoromethoxyphenyl)-3-(2-pyridyl)-1,2,4-oxadiazole, 5-(3-methoxycarbonyl-5-trifluoromethoxyphenyl)-3-(2-pyridyl)-1,2,4-oxadiazole, 5-(3-cyano-5-trifluoromethoxyphenyl)-3-(5-fluoro-pyrid-2-yl)-1,2,4-oxadiazole, 3-(5-cyano-pyrid-2-yl)-5-(3-cyano-5-trifluoromethoxyphenyl)-1,2,4-oxadiazole, 3-(3-cyano-5-dimethylaminophenyl)-5-(5-fluoro-pyrid-2-yl)-1,2,4-oxadiazole, 5-(5-chloro-pyrid-2-yl)-3-(3-cyano-5-dimethylaminophenyl)-1,2,4-oxadiazole, 5-(5-chloro-pyrid-2-yl)-3-(3-cyano-5-methoxyphenyl)-1,2,4-oxadiazole, 5-(5-chloro-pyrid-2-yl)-3-(6-cyano-4-methoxy-pyrid-2-yl)-1,2,4-oxadiazole, 5-(5-chloro-pyrid-2-yl)-3-(6-cyano-4-hydroxy-pyrid-2-yl)-1,2,4-oxadiazole, 5-(5-chloro-pyrid-2-yl)-3-(3-cyano-5-trifluoromethoxyphenyl)-1,2,4-oxadiazole, 5-(5-chloro-pyrid-2-yl)-3-(3-cyanophenyl)-1,2,4-oxadiazole, 5-(5-chloro-pyrid-2-yl)-3-(3-cyano-5-methylphenyl)-1,2,4-oxadiazole, 5-(5-chloro-pyrid-2-yl)-3-(3-cyano-5-fluorophenyl)-1,2,4-oxadiazole, 3-(3-cyano-5-trifluoromethoxyphenyl)-5-(2-pyridyl)-1,2,4-oxadiazole, 3-(3-fluoro-5-methoxyphenyl)-5-(2-pyridyl)-1,2,4-oxadiazole, 3-(3-cyanophenyl)-5-(5-fluoro-pyrid-2-yl)-1,2,4-oxadiazole, 3-(3-cyano-5-trifluoromethoxyphenyl)-5-(5-fluoro-pyrid-2-yl)-1,2,4-oxadiazole, 3-(3-fluoro-5-methoxyphenyl)-5-(5-fluoropyrid-2-yl)-1,2,4-oxadiazole, 3-(3,5-dimethoxyphenyl)-5-(5-fluoropyrid-2-yl)-1,2,4-oxadiazole, 3-(3-fluoro-5-(1H-imidazol-1-yl)phenyl)-5-(2-pyridyl)-1,2,4-oxadiazole, 3-(5-fluoro-2-pyridyl)-5-(3-fluoro-5-(3-pyridyl)phenyl)-1,2,4-oxadiazole, 3-(5-fluoro-2-pyridyl)-5-(3-bromo-5-(3-pyridyl)phenyl)-1,2,4-oxadiazole, 3-(5-fluoro-2-pyridyl)-5-(3-fluoro-5-methoxyphenyl)-1,2,4-oxadiazole, 3-(pyrid-2-yl)-5-(3-cyano-5-thiomethylphenyl)-1,2,4-oxadiazole, 3-(5-fluoro-pyrid-2-yl)-5-(3-cyano-5-thiomethylphenyl)-1,2,4-oxadiazole, 3-(pyrid-2-yl)-5-(3-fluoro-5-thiomethylphenyl)-1,2,4-oxadiazole, 3-(pyrid-2-yl)-5-(3-fluoro-5-thiomethylsulphoxidephenyl)-1,2,4-oxadiazole, 3-(pyrid-2-yl)-5-(3-fluoro-5-thioethylphenyl)-1,2,4-oxadiazole, 3-(pyrid-2-yl)-5-(5-fluoro-3-thiotertbutylphenyl)-1,2,4-oxadiazole, 3-(5-fluoropyrid-2-yl)-5-(3-cyano-5-methylphenyl)-1,2,4-oxadiazole, 3-(5-cyanopyrid-2-yl)-5-(3-cyano-5-thiomethylphenyl)-1,2,4-oxadiazole, 3-(5-fluoropyrid-2-yl)-5-(4-cyano-2-thienyl)-1,2,4-oxadiazole, 3-(2-pyridyl)-5-(3-cyano-5-trifluoromethylphenyl)-1,2,4-oxadiazole, 3-(5-fluoro-2-pyridyl)-5-(3-cyano-5-trifluoromethylphenyl)-1,2,4-oxadiazole, 3-(5-fluoro-pyrid-2-yl)-5-(3-(3-pyridyl)phenyl)-1,2,4-oxadiazole, 5-(3-methyl-5-(3-pyridyl)-pyrid-4-yl)-3-(2-pyridyl)-1,2,4-oxadiazole, 5-(3-methoxy-5-(3-pyridyl)-pyrid-4-yl)-3-(2-pyridyl)-1,2,4-oxadiazole, 5-(2-pyridyl)-3-(5-(3-pyridyl)-pyrid-3-yl)-1,2,4-oxadiazole, 5-(2-pyridyl)-3-(3-(3-pyridyl)-phenyl)-1,2,4-oxadiazole, 5-(5-fluoro-pyrid-2-yl)-3-(3-fluoro-5-(3-pyridyl)-phenyl)-1,2,4-oxadiazole, 3-(2-pyridyl)-5-(3-carboxy-5-methoxyphenyl)-1,2,4-oxadiazole, 3-(2-pyridyl)-5-(3-(carboxamido)-5-methoxyphenyl)-1,2,4-oxadiazole, 3-(2-pyridyl)-5-(3-cyano-5-(2-N,N-dimethylaminoethoxy)phenyl)-1,2,4-oxadiazole, 3-(2-pyridyl)-5-(3-cyano-5-(N,N-dimethylaminopropoxy)phenyl)-1,2,4-oxadiazole, 3-(2-pyridyl)-5-(3-cyano-5-(2 aminoethoxy)phenyl)-1,2,4-oxadiazole, 3-(2-pyridyl)-5-(3-cyano-5-(2-hydroxyethoxy)phenyl)-1,2,4-oxadiazole, 3-(2-pyridyl)-5-(3-cyano-5-isopropoxyphenyl)-1,2,4-oxadiazole, 3-(2-pyridyl)-5-(3-cyano-5-ethoxyphenyl)-1,2,4-oxadiazole, 3-(2-pyridyl)-5-(3-cyano-5-(2,2,2-trifluoroethoxy)phenyl)-1,2,4-oxadiazole, 3-(2-pyridyl)-5-(3-cyano-5-cyclopropylmethoxyphenyl)-1,2,4-oxadiazole, 3-(2-pyridyl)-5-(3-amino-5-cyanophenyl)-1,2,4-oxadiazole, 3-(5-fluoropyrid-2-yl)-5-(3-amino-5-cyanophenyl)-1,2,4-oxadiazole, 3-(2-pyridyl)-5-(3-cyano-5-(trifluoromethylsulfonyloxy)phenyl)-1,2,4-oxadiazole, 3-(2-pyridyl)-5-(3-cyano-5-(2-methoxy-2-oxoethoxy)phenyl)-1,2,4-oxadiazole, 3-(2-pyridyl)-5-(3-cyano-5-(2-tert-butoxy-2-oxoethoxy)phenyl)-1,2,4-oxadiazole, 3-(2-pyridyl)-5-(3-cyano-5-(methoxymethoxy)phenyl)-1,2,4-oxadiazole, 3-(2-pyridyl)-5-(3-cyano-5-(methoxyethoxy)phenyl)-1,2,4-oxadiazole, 3-(5-fluoropyrid-2-yl)-5-(3-cyano-5-cyclopentylaminophenyl)-1,2,4-oxadiazole, 3-(2-pyridyl)-5-(3-cyano-5-hexyloxyphenyl)-1,2,4-oxadiazole, 3-(2-pyridyl)-5-(3-cyano-5-(dimethylamino)carbonylphenyl)-1,2,4-oxadiazole, 3-(5-fluoropyrid-2-yl)-5-(3-cyano-5-ethylaminophenyl)-1,2,4-oxadiazole, 3-(5-fluoropyrid-2-yl)-5-(3-cyano-5-diethylaminophenyl)-1,2,4-oxadiazole, 3-(5-fluoro-pyrid-2-yl)-5-(3-fluoro-5-(1H-tetraazol-5-yl)-phenyl)-1,2,4-oxadiazole, 5-(3-fluoro-5-(1-methyl-1H-tetraazol-5-yl)-phenyl)-3-(5-fluoro-pyrid-2-yl)-1,2,4-oxadiazole, 3-(5-fluoro-2-pyridyl)-5-(3-(1-benzyl-1,2,5,6-tetrahydropyridin-3-yl)-5-fluorophenyl)-1,2,4-oxadiazole, 3-(5-fluoro-2-pyridyl)-5-(3-fluoro-5-(1H-imidazol-4-yl)phenyl)-1,2,4-oxadiazole, 1-(3-cyanophenyl)-4-(5-fluoro-2-pyridyl)-1H-imidazole, 3-(2-pyridyl)-5-(3-(1H-imidazol-1-yl)-5-thiomethoxyphenyl)-1,2,4-oxadiazole, 3-(3-cyano-5-(1H-imidazol-1-yl)phenyl)-5-(2-pyridyl)-1,2,4-oxadiazole, 3-(2-pyridyl)-5-(3-cyano-5-(1H-imidazol-1-yl)phenyl)-1,2,4-oxadiazole, 2-(3-iodophenyl)-4-(pyridin-2-yl)-1,3-thiazole, 3-(2-pyridyl)-2-(3-cyanophenyl)-furan, 3-(5-fluoro-2-pyridyl)-2-(3-cyanophenyl)-furan, 3-(5-chloro-2-pyridyl)-2-(3-cyanophenyl)-furan, 2-(2-pyridyl)-5-(5-fluoro-3-(1-imidazolyl)phenyl)-furan, 3-(5-(2-pyridyl)-2-furyl)-benzonitrile, 3-(5-(5-chloro-2-pyridyl)-2-furyl)-benzonitrile, 3-(5-(5-cyano-2-pyridyl)-2-furyl)-benzonitrile, 3-(5-(5-fluoro-2-pyridyl)-2-furyl)-benzonitrile, 3-fluoro-5-(5-(2-pyridyl)-2-furyl)-benzonitrile, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyano-5-fluorophenyl)-4-(2-pyridyl)-1,3-oxazole, 2-(5-allyloxy-3-cyanophenyl)-4-(5-fluoropyrid-2-yl)-1,3-oxazole, 2-(3-cyano-5-methoxyphenyl)-4-(pyrid-2-yl)-1,3-oxazole, 2-(3-cyano-5-methoxyphenyl)-4-(5 fluoropyrid-2-yl)-1,3-oxazole, 2-(3-cyano-5-n-propyloxyphenyl)-4-(5-fluoropyrid-2-yl)-1,3-oxazole, and 2-(3-cyano-5-methoxyphenyl)-4-(4-pyrid-2-yl)-5-chloro-1,3-oxazole.

The compounds of formula II above may be prepared as described in WO01/12627 A1 and WO02/068417 A2.

The compounds of formula II above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

A further aspect of the invention is the use of a compound of formula II for the manufacture of a medicament for the prevention of weight gain.

Still a further aspect of the invention is the use of a compound of formula II for the manufacture of a medicament for modulation of appetite.

Still a further aspect of the invention is the use of a compound of formula II for the manufacture of a medicament for maintenance of weight loss.

Still a further aspect of the invention is a method for the treatment or prevention of obesity, whereby a pharmaceutically and pharmacologically effective amount of a compound of formula II is administered to a subject in need of such treatment.

A further aspect of the invention is the use of a compound of formula II for the manufacture of a medicament for the prevention of weight regain.

Yet another aspect of the invention is a method for the prevention of weight gain, whereby a pharmaceutically and pharmacologically effective amount of a compound of formula II is administered to a subject in need of such prevention.

Still a further aspect of the invention is a method for the modulation of appetite, whereby a pharmaceutically and pharmacologically effective amount of a compound of formula II is administered to a subject in need of such treatment.

Still a further aspect of the invention is a method for the maintenance of weight loss, whereby a pharmaceutically and pharmacologically effective amount of a compound of formula II is administered to a subject in need of such maintenance.

Still a further aspect of the invention is a method for prevention of weight regain, whereby a pharmaceutically and pharmacologically effective amount of a compound of formula II is administered to a subject in need of such prevention.

A compound of formula II may optionally be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions. Examples of such compounds are orlistat, rimonabant or a monoamine reuptake inhibitor, such as sibutramine hydrochloride monohydrate.

Obesity is defined as a BMI (body mass index) over 30 kg/m². Patients with a BMI between 25 and 29.9 are considered overweight, but not obese. BMI is calculated by dividing weight in kilograms by height in metres squared.

A further group of subjects that would benefit from the treatment according to the present invention are subjects having a BMI over 27 kg/m² and with existing co-morbidities.

### Pharmaceutical Formulations

For clinical use, the compounds of formula II are in accordance with the present invention suitably formulated into pharmaceutical formulations for oral administration. Also rectal, parenteral or any other route of administration may be contemplated by the skilled man in the art of formulations. Thus, the compounds of formula II are formulated with at least one pharmaceutically and pharmacologically acceptable carrier or adjuvant. The carrier may be in the form of a solid, semi-solid or liquid diluent.

In the preparation of oral pharmaceutical formulations in accordance with the invention, the compound of formula II to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of the active compound or compounds of the invention, vegetable oil, fat, or other suitable vehicle for soft gelatine capsules. Hard gelatine capsules may contain the active compound in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains the active substance in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the active compound and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, the compound of formula II may be administered once or twice daily, depending on the severity of the patient's condition.

### Biological Evaluation

### Pharmacodynamic Effect in Rat

Male Wistar-Hanover rats (Charles River, 300-350 grams) are acclimated to individually housing in conventional cages (Makrolon III) with 12:12 hour light-dark photoperiod in a temperature (20-22° C.) and humidity (40-60%) controlled room. R-3 lab chow (Lactanin, Vadstena, Sweden) and tap water from bottles is allowed ad libitum. The day before experiments, animals are weighed and food (but not water) is removed. 20 hours later (on the experiment day), animals are weighed and compound or vehicle (3-10% DMA depending on compound formulation) is administered systemically. Animals are returned to their home cages and given access to a weighed amount of food. This food is then re-weighed after 1, 2, 4, 6 and 24 hours, and food consumption calculated by the difference from initial food weight.

Animals are further weighed at the 24-hour timepoint, and change in body weight over the treatment period is calculated.

### Pharmacodynamic Effect in Mouse

Female C57Bl6 mice (19-21 g) are singly housed for 7-days with ad libitum access to a “bland-paste” made from normal laboratory chow (R-3 Lactanin, Vadstena, Sweden) or to a “palatable-paste” of similar consistency containing oatmeal, butter, sugar, cocoa powder, cocoa butter and peanut butter. The day before the experimental day, food is removed for 12 hours. O the experiment day, animals are weighed and compound or vehicle is administered. Animals are returned to their home cages and given access to weighed amounts of both bland and palatable pastes. This food is then re-weighed 2, and optionally 4 and 6 hours later, and consumption of each food type calculated by the difference from initial food weight. Animals are further weighed at 24 hours after administration, and change in body weight over the treatment period is calculated.

### Disease Efficacy in Mouse

The ability of the compounds to decrease body weight in cafeteria diet-induced obese mice is used as the main in vivo ‘end-point’ test. Female C57Bl/6J mice are given ad libitum access to calorie-dense ‘cafeteria’ diet (soft chocolate/cocoa-type pastry, chocolate, fatty cheese and nougat) and standard lab chow for 8-10 weeks. Compounds to be tested are then administered systemically (intravenous, intraperitoneal, subcutaneous or per oral) once daily for a minimum of 5 days, and their body weights are monitored on a daily basis. Simultaneous assessment of adiposity is carried out by means of DEXA (Dual Energy X-Ray Absorptiometry) imaging at baseline and termination of the study. Blood sampling is also carried out to assay changes in obesity-related plasma markers.

### Results

The pharmacodynamic effect of 3-[3-(5-fluoropyridin-2-yl)-1,2,4-oxadiazol-5-yl]-5-(methoxymethyl)benzonitrile was investigated in mouse as described above.

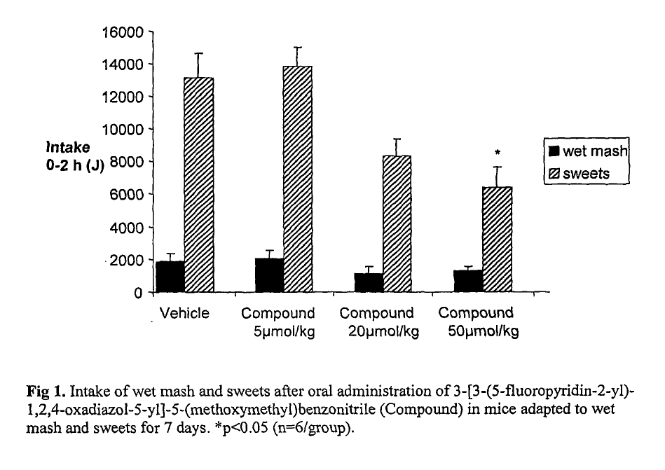

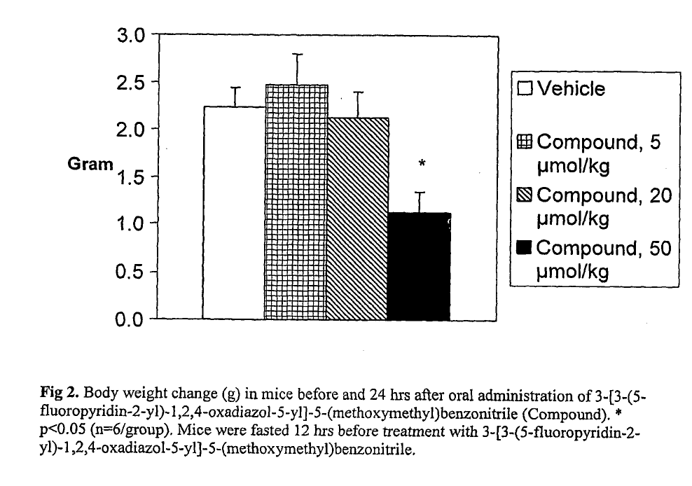

3-[3-(5-fluoropyridin-2-yl)-1,2,4-oxadiazol-5-yl]-5-(methoxymethyl)benzonitrile decreased intake of sweets measured 2 hours after treatment in a dose-related manner, whereas no effect on normal laboratory chow was found, indicating an effect of 3-[3-(5-fluoropyridin-2-yl)-1,2,4-oxadiazol-5-yl]-5-(methoxymethyl)benzonitrile on high palatable food (FIG. 1). Moreover, animals receiving 3-[3-(5-fluoropyridin-2-yl)-1,2,4-oxadiazol-5-yl]-5-(methoxymethyl)benzonitrile had a slower body weight recovery after fasting compared to vehicle treated animals (FIG. 2).

Generally, compounds useful in accordance with the present invention tested in the animal models above cause a decrease in the intake of sweets of up to 50%, compared to vehicle treated animals.

1. Use of a compound of formula II, 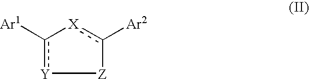
wherein X, Y, and Z are independently selected from the group consisting of N, O, S, C, and CO wherein at least one of X, Y, and Z is a heteroatom; Ar¹ and Ar² are independently selected from the group consisting of a heterocyclic or fused heterocyclic moiety containing 1 to 4 heteroatoms selected from the group consisting of N, O, and S and an aromatic moiety selected from the group consisting of phenyl, benzyl, 1-naphthyl, 2-naphthyl, fluorenyl, anthrenyl, indenyl, phenanthrenyl, and benzonaphthenyl, wherein the Ar¹ and Ar² moieties are optionally substituted with one or more moieties selected from the group consisting of —F, —Cl, —Br, —I, —OR, —SR, —SOR, —SO₂R, —SO₂NRR′, —OCOR, —OCONRR′, —NRCOR′, —NRCO₂R′, —CN, —NO₂, —CO₂R, —CONRR′, —C(O)R, —CH(OR)R′, —CH₂(OR), —R, and -A-(CH₂)—NRR′; wherein R or R′ is selected from the group consisting of H, CF₃, C₁-C₁₀ alkyl, cycloalkyl, alkyl-aryl, alkyl-heteroaryl, heterocycloalkyl, aryl and where R and R′ may combine to form a ring, and A is defined as CH₂, O, NH, S, SO, SO₂ and n is 1, 2, 3, or 4;
or a pharmaceutically acceptable salt or an optical isomer thereof, for the manufacture of a medicament for the treatment or prevention of obesity.

2. Use of a compound of formula II according to claim 1,
wherein X and Y are N;
Z is O;
Ar¹ is 2-pyridyl and Ar² is phenyl, wherein at least one of the Ar¹ and Ar² moieties are substituted with one or more moieties selected from the group consisting of —F, —Cl, —Br, —I, SR, —SOR, —SO₂R, SO₂NRR′, —OCOR, —OCONRR′,
—NRCOR′, —NRCO₂R′, —CN, —CO₂R, —CONRR′, —C(O)R, —CH₂(OR)R′, —CH₂(OR), and —R;
wherein R or R′ is selected from the group consisting of H, CF₃, C₁-C₁₀ alkyl, cycloalkyl, alkyl-aryl, heterocycloalkyl, aryl and where R and R′ may combine to form a ring;
or a pharmaceutically acceptable salt or an optical isomer thereof, for the manufacture of a medicament for the treatment or prevention of obesity.

3. Use according to claim 2, wherein Ar² is substituted by one or more moieties selected from the group consisting of CF₃, Cl, F, Br, CH₃, SCH₃ and CN.

4. Use according to claim 2, wherein Ar¹ is substituted by one or more moieties selected from the group consisting of CF₃, F and Cl.

5. Use of a compound of formula II as defined in claim 1, or a pharmaceutically acceptable salt or an optical isomer thereof, for the manufacture of a medicament for the prevention of weight gain.

6. Use of a compound of formula II as defined in claim 1, or a pharmaceutically acceptable salt or an optical isomer thereof, for the manufacture of a medicament for the modulation of appetite.

7. Use according to claim 1, wherein the compound of formula II is selected from the group of compounds consisting of 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(3-methoxyphenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 2-(3-chlorophenyl)-4-(pyridin-2-yl)-1,3-oxazole and 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole and pharmaceutically acceptable salts or optical isomers thereof.

8. Use according to claim 1, wherein the compound of formula II is selected from the group of compounds consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methylphenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-(3-chlorophenyl)-4-(pyridin-2-yl)-1,3-oxazole and 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 2-(3-bromophenyl)-4-(pyridin-2-yl)-1,3-oxazole, 2-(3-cyanophenyl)-4-(pyridin-2-yl)-1,3-oxazole, 5-(3-hydroxyphenyl)-3-(pyridine-2-yl)-1,2-oxazole, 5-(3-cyanophenyl)-3-(pyridin-2-yl)-1,2-oxazole, 3-(2-pyridyl)-5-(3-iodophenyl)-1,2,4-triazole, 3-(2-pyridyl)-5-(5-chloro-2-aminophenyl)-1,2,4-oxadiazole and pharmaceutically acceptable salts or optical isomers thereof.

9. Use according to claim 1, wherein the compound of formula II is selected from the group of compounds consisting of 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole, 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methyl phenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-[3-chlorophenyl]-4-[pyridin-2-yl]-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole and pharmaceutically acceptable salts or optical isomers thereof.

10. Use according to claim 1, wherein the compound of formula II is selected from the group of compounds consisting of 3-(5-Methyl-pyrid-2-yl)-5-(3-cyanophenyl)-1,2,4-oxadiazole, 3-(5-Cyano-pyrid-2-yl)-5-(3-cyanophenyl)-1,2,4-oxadiazole, 3-(2-Pyridyl)-5-(5-bromo-2-methoxyphenyl)-1,2,4-oxadiazole, 3-(2-Pyridyl)-5-(5-bromo-2-fluorophenyl)-1,2,4-oxadiazole, 3-(2-Pyridyl)-5-(5-cyano-2-fluorophenyl)-1,2,4-oxadiazole, 3-(2-Pyridyl)-5-(5-bromopyrid-3-yl)-1,2,4-oxadiazole, 3-(2-Pyridyl)-5-(5-chloro-pyrid-3-yl)-1,2,4-oxadiazole, 3-(5-Cyanopyrid-2-yl)-5-(5-bromo-pyrid-3-yl)-1,2,4-oxadiazole, 3-(5-Fluoropyrid-2-yl)-5-(5-bromo-pyrid-3-yl)-1,2,4-oxadiazole, 3-(2-Pyridyl)-5-(2-thiomethoxy-pyrid-3-yl)-1,2,4-oxadiazole, 3-(2-Pyridyl)-5-(5-methylpyrid-3-yl)-1,2,4-oxadiazole, 3-(2-Pyridyl)-5-(5-methoxypyrid-3-yl)-1,2,4-oxadiazole, 3-(2-Pyridyl)-5-(3-cyano-5-methylphenyl)-1,2,4-oxadiazole, 3-(2-Pyridyl)-5-(3-fluoro-5-bromophenyl)-1,2,4-oxadiazole, 3-(2-Pyridyl)-5-(3-iodo-5-bromophenyl)-1,2,4-oxadiazole, 3-(5-Fluoro-2-pyridyl)-5-(3-fluoro-5-bromophenyl)-1,2,4-oxadiazole, 3-(2-Pyridyl)-5-(3-iodo-5-(methylphenylester)-1,2,4-oxadiazole, 3-(2-Pyridyl)-5-(3-methoxy-5-(methoxycarbonyl)phenyl)-1,2,4-oxadiazole, 3-(2-Pyridyl)-5-(3-bromo-5-cyanophenyl)-1,2,4-oxadiazole, 3-(2-Pyridyl)-5-(5-cyano-3-iodophenyl)-1,2,4-oxadiazole, 3-(5-Cyano-2-pyridyl)-5-(3-bromophenyl)-1,2,4-oxadiazole, 3-(5-Cyano-2-pyridyl)-5-(3-cyano-5-fluorophenyl)-1,2,4-oxadiazole, 3-(5-Cyano-2-pyridyl)-5-(3-bromo-5-fluorophenyl)-1,2,4-oxadiazole, 3-(2-Pyridyl)-5-(5-cyano-2-methoxyphenyl)-1,2,4-oxadiazole, 3-(2-Pyridyl)-5-(2-cyano-5-methoxyphenyl)-1,2,4-oxadiazole, 3-(2-Pyridyl)-5-(5-cyano-pyrid-3-yl)-1,2,4-oxadiazole, 3-(2-Pyridyl)-5-(3-cyano-5-(methoxycarbonyl)phenyl)-1,2,4-oxadiazole, 3-(2-Pyridyl)-5-(5-phenyl-pyrid-3-yl)-1,2,4-oxadiazole, 3-(2-Pyridyl)-5-(3-cyano-5-methoxyphenyl)-1,2,4-oxadiazole, 3-(2-Pyridyl)-5-(3-cyano-5-hydroxyphenyl)-1,2,4-oxadiazole, 3-(2-Pyridyl)-5-(3-cyano-5-propoxyphenyl)-1,2,4-oxadiazole, 2-(3-Cyanophenyl)-4-(pyridin-2-yl)-1,3-thiazole, 2-((3-Bromo-5-iodophenyl)-4-pyridin-2-yl)-1,3-oxazole, 2-(2-Pyridyl)-5-(3-iodophenyl)-1,3,4-oxadiazole, 2-(2-Pyridyl)-5-(3-cyanophenyl)-1,3,4-oxadiazole, 2-(2-Pyridyl)-5-(3-cyanophenyl)-1,3,4-triazole, 3-(5-Chloropyrid-2-yl)-5-(3-cyano-5-fluorophenyl)-1,2,4-oxadiazole, 3-(5-Chloropyrid-2-yl)-5-(3-cyano-5-chlorophenyl)-1,2,4-oxadiazole, 3-(5-Chloropyrid-2-yl)-5-(3-chloro-5-fluorophenyl)-1,2,4-oxadiazole, 3-(5-Chloropyrid-2-yl)-5-(3-cyano-5-methoxyphenyl)-1,2,4-oxadiazole, 3-(5-Fluoropyrid-2-yl)-5-(3-cyano-5-chlorophenyl)-1,2,4-oxadiazole, 3-(5-Fluoropyrid-2-yl)-5-(3-fluoro-5-chlorophenyl)-1,2,4-oxadiazole, 3-(5-Fluoropyrid-2-yl)-5-(3-cyano-5-methoxyphenyl)-1,2,4-oxadiazole, 3-(5-Cyanopyrid-2-yl)-5-(3-cyano-5-chlorophenyl)-1,2,4-oxadiazole, 3-(5-Cyanopyrid-2-yl)-5-(3-fluoro-5-chlorophenyl)-1,2,4-oxadiazole, 3-(5-Cyanopyrid-2-yl)-5-(3-cyano-5-methoxyphenyl)-1,2,4-oxadiazole, 3-(5-Fluoropyrid-2-yl)-5-(3,5-di-cyanophenyl)-1,2,4-oxadiazole, 3-(3-(4-Dimethylaminobutoxy)-pyrid-2-yl)-5-(3-cyano-5-fluorophenyl)-1,2,4-oxadiazole, 3-(3-(5-Dimethylaminopentyloxy)-pyrid-2-yl)-5-(3-Cyano-5-fluorophenyl)-1,2,4-oxadiazole, and [3-(3-(6-Dimethylaminohexyloxy)-pyrid-2-yl)-5-(3-cyano-5-fluorophenyl)-1,2,4- and pharmaceutically acceptable salts or optical isomers thereof.

11. Use according to claim 1, wherein the compound of formula II is selected from the group of compounds consisting of 3-(2-Pyridyl)-5-(3-allyloxy-5-(methoxycarbonyl)phenyl)-1,2,4-oxadiazole, 3-(2-Pyridyl)-5-(3-N,N-dimethylaminophenyl)-1,2,4-oxadiazole), 3-(2-Pyridyl)-5-(3-cyano-5-(4-pyridyl)phenyl)-1,2,4-oxadiazole, 3-(2-Pyridyl)-5-[2-methoxy-5-(4-pyridyl)phenyl]-1,2,4-oxadiazole, 3-(2-pyridyl)-5-[2-fluoro-5-(4-pyridyl)phenyl]-1,2,4-oxadiazole, 3-(2-Pyridyl)-5-(3-fluoro-5-(4-pyridyl)phenyl)-1,2,4-oxadiazole, 3-(2-Pyridyl)-5-(3-fluoro-5-(3-pyridyl)phenyl)-1,2,4-oxadiazole, 3-(2-Pyridyl)-5-[2-fluoro-5-(3-pyridyl)phenyl]-1,2,4-oxediazole, 3-(2-Pyridyl)-5-[2-methoxy-5-(3-pyridyl)phenyl]-1,2,4-oxadiazole, 3-(2-Pyridyl)-5-(3-cyano-5-(3-pyridyl)phenyl)-1,2,4-oxadiazole, 3-(5-Fluoro-2-pyridyl)-5-(3-fluoro-5-(3-pyridyl)phenyl)-1,2,4-oxadiazole, 3-(2-Pyridyl)-5-[5-(3-pyridyl-pyrid-3-yl)]-]-1,2,4-oxadiazole, 3-(5-Fluoropyrid-2-yl)]-5-[5-(3-pyridyl-pyrid-3-yl)]-3-1,2,4-oxadiazole, 3-(5-Cyanopyrid-2-yl)-5-(3-(pyrid-3-yl)phenyl)-1,2,4-oxadiazole, to 3-(5-Cyanopyrid-2-yl)-5-(3-fluoro-5-(pyrid-3-yl)phenyl)-1,2,4-oxadiazole, 3-(2-Pyridyl)-5-(3-cyano-5-(2-pyridyl)phenyl)-1,2,4-oxadiazole, 3-(2-Pyridyl)-5-[2-methoxy-5-(2-pyridyl)phenyl]-1,2,4-oxediazole, 3-(2-Pyridyl)-5-[2-fluoro-5-(2-pyridyl)phenyl]-1,2,4-oxadiazole, 3-(2-Pyridyl)-5-[(3-(3-fluorophenyl)-5-fluorophenyl)]-1,2,4-oxadiazole, 3-(2-Pyridyl)-5-(3-cyano-5-(3-thiophene)phenyl)-1,2,4-oxadiazole, 3-(2-Pyridyl)-5-[5-(3-thienyl)-pyrid-3-yl]-1,2,4-oxadiazole, 3-(2-Pyridyl)-5-[5-3-furyl)-pyrid-3-yl]-1,2,4-oxadiazole, 3-(2-Pyridyl)-5-[5-(3-methoxyphenyl)-pyrid-3-yl]-1,2,4-oxadiazole, 3-(2-Pyridyl)-5-(3-cyano-5-(5-pyrimidyl)phenyl)-1,2,4-oxadiazole, 3-(2-Pyridyl)-5-(3-cyano-5-(3-aminophenyl)phenyl)-1,2,4-oxadiazole, 3-(2-Pyridyl)-5-(3-cyano-5-(3-fluorophenyl)phenyl)-1,2,4-oxediazole, 3-(2-Pyridyl)-5-[5-(5-pyrimidyl)-pyrid-3-yl]-1,2,4-oxadiazole, 3-(2-Pyridyl)-5-(3-aminomethyl-5-cyanophenyl)-1,2,4-oxadiazole, 3-(2-Pyridyl)-5-[5[(2-propenyl)-pyrid-3-yl]-1,2,4-oxediazole, 3-(2-Pyridyl)-5-(3-cyano-5-vinylphenyl)-1,2,4-oxadiazole, 3-(2-Pyridyl)-5-(3-cyano-5-(2-hydroxyethyl)phenyl)-1,2,4-oxadiazole, 3-(2-Pyridyl)-5-(3-cyano-5-(2,3-dichloropropoxy)phenyl)-1,2,4-oxadiazole, 3-(2-Pyridyl)-5-(3-allyloxy-5-carboxyphenyl)-1,2,4-oxadiazole, 3-(2-Pyridyl)-5-(3-allyloxy-5-cyanophenyl)-1,2,4-oxadiazole, 3-(2-Pyridyl)-5-(5-cyano-3-[3-hydroxypropyn-1-yl]phenyl)-1,2,4-oxadiazole, 3-(2-Pyridyl)-5-(2-N-methylaminophenyl)-1,2,4-oxadiazole, and 3-(2-Pyridyl)-5-[5-(3-N-benzyl-1,2,5,6, tetrahydropyridine)-pyrid-3-yl]-1,2,4-oxadiazole.

12. Use according to claim 1, wherein the compound of formula II is selected from the group of compounds consisting of 3-(2-pyridyl)-5-(3-fluorophenyl)-1,2,4-oxadiazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 3-(2-pyridyl)-5-(5-chloro-2-aminophenyl)-1,2,4-oxadiazole, 5-[3-hydroxyphenyl]-3-[pyridin-2-yl]-1,2-oxazole, 5-[3-cyanophenyl]-3-[pyridin-2-yl]-1,2-oxazole, 3-(2-pyridyl)-5-(3-chlorophenyl)-1,2,4-triazole, 3-(2-pyridyl)-5-(3-iodophenyl)-1,2,4-triazole, 3-(2-pyridyl)-5-(5-hydroxypyrid-3-yl)-1,2,4-oxadiazole, 3-(5-fluoropyrid-2-yl)-5-(5-fluoro-3-(thiomethyl)phenyl)-1,2,4-oxadiazole, 5-(2-pyridyl)-3-[3-(1H-imidazol-1-yl)-5-fluorophenyl)]-1,2,4-oxadiazole, 3-(2-pyridyl)-5-(3-cyano-5-trifluoromethylphenyl)-1,2,4-oxadiazole, 3-(5-fluoro-2-pyridyl)-5-(3-fluoro-5-trifluoromethylphenyl)-1,2,4-oxadiazole, 3-(5-cyanopyrid-2-yl)-5-(3-alloxy-5-cyanophenyl)-1,2,4-oxadiazole, 3-(5-fluoropyrid-2-yl)-5-(3-alloxy-5-cyanophenyl)-1,2,4-oxadiazole, 3-(5-cyanopyrid-2-yl)-5-(3-cyano-5-propoxyphenyl)-1,2,4-oxadiazole, 3-(5-fluoropyrid-2-yl)-5-(3-cyano-5-propoxyphenyl)-1,2,4-oxadiazole, 3-(2-pyridyl)-5-(3-cyano-5-nitrophenyl)-1,2,4-oxadiazole, 3-(5-fluoropyrid-2-yl)-5-(3-cyano-5-nitrophenyl)-1,2,4-oxadiazole, 3-(5-fluoropyrid-2-yl)-5-(3-cyano-5-dimethylaminophenyl)-1,2,4-oxadiazole, 3-(5-fluoropyrid-2-yl)-5-(3-cyano-5-(2-methoxyethoxy)phenyl)-1,2,4-oxadiazole, 3-(5-fluoropyrid-2-yl)-5-(3-cyano-5-(1H-imidazol-1-ylmethyl)phenyl)-1,2,4-oxadiazole, 3-(2-pyridyl)-5-(3-cyano-5-(methoxymethyl)phenyl)-1,2,4-oxadiazole, 3-(3-cyano-5-methoxyphenyl)-5-(2-pyridyl)-1,2,4-oxadiazole, 3-(3-cyano-5-methoxyphenyl)-5-(5-fluoropyrid-2-yl)-1,2,4-oxadiazole, 3-(5-fluoropyrid-2-yl)-5-(3-cyano-5-ethoxyphenyl)-1,2,4-oxadiazole, 3-(5-cyanopyrid-2-yl)-5-(3-cyano-5-ethoxyphenyl)-1,2,4-oxadiazole, 3-(5-chloropyrid-2-yl)-5-(3-allyloxy-5-cyanophenyl)-1,2,4-oxadiazole, 3-(5-chloropyrid-2-yl)-5-(3-cyano-5-propoxyphenyl)-1,2,4-oxadiazole, 3-(5-chloropyrid-2-yl)-5-(3-cyano-5-ethoxyphenyl)-1,2,4-oxadiazole, 3-(5-fluoropyrid-2-yl)-5-(3-cyano-5-hexyloxyphenyl)-1,2,4-oxadiazole, 3-(5-fluoropyrid-2-yl)-5-(3-cyano-5-(methoxymethyl)phenyl)-1,2,4-oxadiazole, 3-(5-fluoropyrid-2-yl)-5-(5-cyano-2-methoxyphenyl)-1,2,4-oxadiazole, 5-(5-cyano-2-methoxyphenyl)-3-(5-cyano-pyrid-2-yl)-1,2,4-oxadiazole, 3-(5-fluoro-pyrid-2-yl)-5-(3-bromophenyl)-1,2,4-oxadiazole, 5-(3-chloro-5-methyl-pyrid-4-yl)-3-(2-pyridyl)-1,2,4-oxadiazole, 5-(3-chloro-5-methoxy-pyrid-4-yl)-3-(2-pyridyl)-1,2,4-oxadiazole, 3-(3-cyano-5-methylphenyl)-5-(2-pyridyl)-1,2,4-oxadiazole, 3-(5-bromo-pyrid-3-yl)-5-(2-pyridyl)-1,2,4-oxadiazole, 3-(3-cyano-5-fluorophenyl)-5-(2-pyridyl)-1,2,4-oxadiazole, 3-(3-iodophenyl)-5-(2-pyridyl)-1,2,4-oxadiazole, 3-(3-cyanophenyl)-5-(2-pyridyl)-1,2,4-oxadiazole, 3-(3-cyano-5-dimethylamino-phenyl)-5-(2-pyridyl)-1,2,4-oxadiazole, 3-(3-cyano-5-methylphenyl)-5-(5-fluoro-pyrid-2-yl)-1,2,4-oxadiazole, 3-(3-cyano-5-fluorophenyl)-5-(5-fluoro-pyrid-2-yl)-1,2,4-oxadiazole, 3-(4-cyanophenyl)-3-(6-cyano-pyrid-2-yl)-1,2,4-oxadiazole, 5-(3-cyano-5-trifluoromethoxyphenyl)-3-(2-pyridyl)-1,2,4-oxadiazole, 5-(3-methoxycarbonyl-5-trifluoromethoxyphenyl)-3-(2-pyridyl)-1,2,4-oxadiazole, 5-(3-cyano-5-trifluoromethoxyphenyl)-3-(5-fluoro-pyrid-2-yl)-1,2,4-oxadiazole, 3-(5-cyano-pyrid-2-yl)-5-(3-cyano-5-trifluoromethoxyphenyl)-1,2,4-oxadiazole, 3-(3-cyano-5-dimethylaminophenyl)-5-(5-fluoro-pyrid-2-yl)-1,2,4-oxadiazole, 5-(5-chloro-pyrid-2-yl)-3-(3-cyano-5-dimethylaminophenyl)-1,2,4-oxadiazole, 5-(5-chloro-pyrid-2-yl)-3-(3-cyano-5-methoxyphenyl)-1,2,4-oxadiazole, 5-(5-chloro-pyrid-2-yl)-3-(6-cyano-4-methoxy-pyrid-2-yl)-1,2,4-oxadiazole, 5-(5-chloro-pyrid-2-yl)-3-(6-cyano-4-hydroxy-pyrid-2-yl)-1,2,4-oxadiazole, 5-(5-chloro-pyrid-2-yl)-3-(3-cyano-5-trifluoromethoxyphenyl)-1,2,4-oxadiazole, 5-(5-chloro-pyrid-2-yl)-3-(3-cyanophenyl)-1,2,4-oxadiazole, 5-(5-chloro-pyrid-2-yl)-3-(3-cyano-5-methylphenyl)-1,2,4-oxadiazole, 5-(5-chloro-pyrid-2-yl)-3-(3-cyano-5-fluorophenyl)-1,2,4-oxadiazole, 3-(3-cyano-5-trifluoromethoxyphenyl)-5-(2-pyridyl)-1,2,4-oxadiazole, 3-(3-fluoro-5-methoxyphenyl)-5-(2-pyridyl)-1,2,4-oxadiazole, 3-(3-cyanophenyl)-5-(5-fluoro-pyrid-2-yl)-1,2,4-oxadiazole, 3-(3-cyano-5-trifluoromethoxyphenyl)-5-(5-fluoro-pyrid-2-yl)-1,2,4-oxadiazole, 3-(3-fluoro-5-methoxyphenyl)-5-(5-fluoropyrid-2-yl)-1,2,4-oxadiazole, 3-(3,5-dimethoxyphenyl)-5-(5-fluoropyrid-2-yl)-1,2,4-oxadiazole, 3-[3-fluoro-5-(1H-imidazol-1-yl)phenyl]-5-(2-pyridyl)-1,2,4-oxadiazole, 3-(5-fluoro-2-pyridyl)-5-(3-fluoro-5-(3-pyridyl)phenyl)-1,2,4-oxadiazole, 3-(5-fluoro-2-pyridyl)-5-(3-bromo-5-(3-pyridyl)phenyl)-1,2,4-oxadiazole, 3-(5-fluoro-2-pyridyl)-5-(3-fluoro-5-methoxyphenyl)-1,2,4-oxadiazole, 3-(pyrid-2-yl)-5-(3-cyano-5-thiomethylphenyl)-1,2,4-oxadiazole, 3-(5-fluoro-pyrid-2-yl)-5-(3-cyano-5-thiomethylphenyl)-1,2,4-oxadiazole, 3-(pyrid-2-yl)-5-(3-fluoro-5-thiomethylphenyl)-1,2,4-oxadiazole, 3-(pyrid-2-yl)-5-(3-fluoro-5-thiomethylsulphoxidephenyl)-1,2,4-oxadiazole, 3-(pyrid-2-yl)-5-(3-fluoro-5-thioethylphenyl)-1,2,4-oxadiazole, 3-(pyrid-2-yl)-5-(5-fluoro-3-thiotertbutylphenyl)-1,2,4-oxadiazole, 3-(5-fluoropyrid-2-yl)-5-(3-cyano-5-methylphenyl)-1,2,4-oxadiazole, 3-(5-cyanopyrid-2-yl)-5-(3-cyano-5-thiomethylphenyl)-1,2,4-oxadiazole, 3-(5-fluoropyrid-2-yl)-5-(4-cyano-2-thienyl)-1,2,4-oxadiazole, 3-(2-pyridyl)-5-(3-cyano-5-trifluoromethylphenyl)-1,2,4-oxadiazole, 3-(5-fluoro-2-pyridyl)-5-(3-cyano-5-trifluoromethylphenyl)-1,2,4-oxadiazole, 3-(5-fluoro-pyrid-2-yl)-5-[3-(3-pyridyl)phenyl]-1,2,4-oxadiazole, 5-[3-methyl-5-(3-pyridyl)-pyrid-4-yl]-3-(2-pyridyl)-1,2,4-oxadiazole, 5-[3-methoxy-5-(3-pyridyl)-pyrid-4-yl]-3-(2-pyridyl)-1,2,4-oxadiazole, 5-(2-pyridyl)-3-[5-(3-pyridyl)-pyrid-3-yl]-1,2,4-oxadiazole, 5-(2-pyridyl)-3-[3-(3-pyridyl)-phenyl]-1,2,4-oxadiazole, 5-(5-fluoro-pyrid-2-yl)-3-[3-fluoro-5-(3-pyridyl)-phenyl]-1,2,4-oxadiazole, 3-(2-pyridyl)-5-(3-carboxy-5-methoxyphenyl)-1,2,4-oxadiazole, 3-(2-pyridyl)-5-(3-(carboxamido)-5-methoxyphenyl)-1,2,4-oxadiazole, 3-(2-pyridyl)-5-(3-cyano-5-(2-N,N-dimethylaminoethoxy)phenyl)-1,2,4-oxadiazole, 3-(2-pyridyl)-5-(3-cyano-5-(N,N-dimethylaminopropoxy)phenyl)-1,2,4-oxadiazole, 3-(2-pyridyl)-5-(3-cyano-5-(2-aminoethoxy)phenyl)-1,2,4-oxadiazole, 3-(2-pyridyl)-5-(3-cyano-5-(2-hydroxyethoxy)phenyl)-1,2,4-oxadiazole, 3-(2-pyridyl)-5-(3-cyano-5-isopropoxyphenyl)-1,2,4-oxadiazole, 3-(2-pyridyl)-5-(3-cyano-5-ethoxyphenyl)-1,2,4-oxadiazole, 3-(2-pyridyl)-5-(3-cyano-5-(2,2,2-trifluoroethoxy)phenyl)-1,2,4-oxadiazole, 3-(2-pyridyl)-5-(3-cyano-5-cyclopropylmethoxyphenyl)-1,2,4-oxadiazole, 3-(2-pyridyl)-5-(3-amino-5-cyanophenyl)-1,2,4-oxadiazole, 3-(5-fluoropyrid-2-yl)-5-(3-amino-5-cyanophenyl)-1,2,4-oxadiazole, 3-(2-pyridyl)-5-(3-cyano-5-(trifluoromethylsulfonyloxy)phenyl)-1,2,4-oxadiazole, 3-(2-pyridyl)-5-(3-cyano-5-(2-methoxy-2-oxoethoxy)phenyl)-1,2,4-oxadiazole, 3-(2-pyridyl)-5-(3-cyano-5-(2-tert-butoxy-2-oxoethoxy)phenyl)-1,2,4-oxadiazole, 3-(2-pyridyl)-5-(3-cyano-5-(methoxymethoxy)phenyl)-1,2,4-oxadiazole, 3-(2-pyridyl)-5-(3-cyano-5-(methoxyethoxy)phenyl)-1,2,4-oxadiazole, 3-(5-fluoropyrid-2-yl)-5-(3-cyano-5-cyclopentylaminophenyl)-1,2,4-oxadiazole, 3-(2-pyridyl)-5-(3-cyano-5-hexyloxyphenyl)-1,2,4-oxadiazole, 3-(2-pyridyl)-5-(3-cyano-5-(dimethylamino)carbonylphenyl)-1,2,4-oxadiazole, 3-(5-fluoropyrid-2-yl)-5-(3-cyano-5-ethylaminophenyl)-1,2,4-oxadiazole, 3-(5-fluoropyrid-2-yl)-5-(3-cyano-5-diethylaminophenyl)-1,2,4-oxadiazole, 3-(5-fluoro-pyrid-2-yl)-5-[3-fluoro-5-(1H-tetraazol-5-yl)-phenyl]-1,2,4-oxadiazole, 5-[3-fluoro-5-(1-methyl-1H-tetraazol-5-yl)-phenyl]-3-(5-fluoro-pyrid-2-yl)-1,2,4-oxadiazole, 3-(5-fluoro-2-pyridyl)-5-(3-(1-benzyl-1,2,5,6-tetrahydropyridin-3-yl)-5-fluorophenyl)-1,2,4-oxadiazole, 3-(5-fluoro-2-pyridyl)-5-(3-fluoro-5-(1H-imidazol-4-yl)phenyl)-1,2,4-oxadiazole, 1-(3-cyanophenyl)-4-(5-fluoro-2-pyridyl)-1H-imidazole, 3-(2-pyridyl)-5-(3-(1H-imidazol-1-yl)-5-thiomethoxyphenyl)-1,2,4-oxadiazole, 3-(3-cyano-5-(1H-imidazol-1-yl)phenyl)-5-(2-pyridyl)-1,2,4-oxadiazole, 3-(2-pyridyl)-5-(3-cyano-5-(1H-imidazol-1-yl)phenyl)-1,2,4-oxadiazole, 2-(3-iodophenyl)-4-(pyridin-2-yl)-1,3-thiazole, 3-(2-pyridyl)-2-(3-cyanophenyl)-furan, 3-(5-fluoro-2-pyridyl)-2-(3-cyanophenyl)-furan, 3-(5-chloro-2-pyridyl)-2-(3-cyanophenyl)-furan, 2-(2-pyridyl)-5-(5-fluoro-3-(1-imidazolyl)phenyl)-furan, 3-(5-(2-pyridyl)-2-furyl)-benzonitrile, 3-(5-(5-chloro-2-pyridyl)-2-furyl)-benzonitrile, 3-(5-(5-cyano-2-pyridyl)-2-furyl)-benzonitrile, 3-(5-(5-fluoro-2-pyridyl)-2-furyl)-benzonitrile, 3-fluoro-5-(5-(2-pyridyl)-2-furyl)-benzonitrile, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyano-5-fluorophenyl)-4-(2-pyridyl)-1,3-oxazole, 2-(5-allyloxy-3-cyanophenyl)-4-(5-fluoropyrid-2-yl)-1,3-oxazole, 2-(3-cyano-5-methoxyphenyl)-4-(pyrid-2-yl)-1,3-oxazole, 2-(3-cyano-5-methoxyphenyl)-4-(5-fluoropyrid-2-yl)-1,3-oxazole, 2-(3-cyano-5-n-propyloxyphenyl)-4-(5-fluoropyrid-2-yl)-1,3-oxazole, and 2-(3-cyano-5-methoxyphenyl)-4-(4-pyrid-2-yl)-5-chloro-1,3-oxazole.

13. A method for the treatment or prevention of obesity, whereby a pharmaceutically and pharmacologically effective amount of a compound of formula II 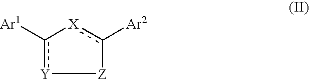
wherein X, Y, and Z are independently selected from the group consisting of N, O, S, C, and CO wherein at least one of X, Y, and Z is a heteroatom; Ar¹ and Ar² are independently selected from the group consisting of a heterocyclic or fused heterocyclic moiety containing 1 to 4 heteroatoms selected from the group consisting of N, O, and S and an aromatic moiety selected from the group consisting of phenyl, benzyl, I-naphthyl, 2-naphthyl, fluorenyl, anthrenyl, indenyl, phenanthrenyl, and benzonaphthenyl, wherein the Ar¹ and Ar² moieties are optionally substituted with one or more moieties selected from the group consisting of —F, —Cl, —Br, —I, —OR, —SR, —SOR, —SO₂R, —SO₂NRR′, —OCOR, —OCONRR′, —NRCOR′, —NRCO₂R′, —CN, —NO₂, —CO₂R, —CONRR′, —C(O)R, —CH(OR)R′, —CH₂(OR), —R, and -A-(CH₂)—NRR′; wherein R or R′ is selected from the group consisting of H, CF₃, C₁-C₁₀ alkyl, cycloalkyl, alkyl-aryl, alkyl-heteroaryl, heterocycloalkyl, aryl and where R and R′ may combine to form a ring, and A is defined as CH₂, O, NH, S, SO, SO₂ and n is 1, 2, 3, or 4;
or a pharmaceutically acceptable salt or an optical isomer thereof, is administered to a subject in need of such treatment or prevention.

14. A method according to claim 13,
wherein, in the compound of formula II,
X and Y are N;
Z is O;
Ar¹ is 2-pyridyl and Ar² is phenyl, wherein at least one of the Ar¹ and Ar² moieties are substituted with one or more moieties selected from the group consisting of —F, —Cl, —Br, —I, SR, —SOR, —SO₂R, SO₂NRR′, —OCOR, —OCONRR′,
—NRCOR′, —NRCO₂R′, —CN, —CO₂R, —CONRR′, —C(O)R, —CH(OR)R′, —CH₂(OR), and —R;
wherein R or R′ is selected from the group consisting of H, CF₃, C₁-C₁₀ alkyl, cycloalkyl, alkyl-aryl, heterocycloalkyl, aryl and where R and R′ may combine to form a ring.

15. A method according to claim 14, wherein Ar² is substituted by one or more moieties selected from the group consisting of CF₃, Cl, F, Br, CH₃, SCH₃ and CN.

16. A method according to claim 14 or 15, wherein Ar¹ is substituted by one or more moieties selected from the group consisting of CF₃, F and Cl.

17. A method for the prevention of weight gain, whereby a pharmaceutically and pharmacologically effective amount of a compound of formula II as defined in claim 13, or a pharmaceutically acceptable salt or an optical isomer thereof, is administered to a subject in need of such prevention.

18. A method for the modulation of appetite, whereby a pharmaceutically and pharmacologically effective amount of a compound of formula II as defined in claim 13, or a pharmaceutically acceptable salt or an optical isomer thereof, is administered to a subject in need of such modulation.

19. A method according to claim 13, wherein the compound of formula II is selected from the group of compounds consisting of 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(3-methoxyphenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 2-(3-chlorophenyl)-4-(pyridin-2-yl)-1,3-oxazole and 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole and pharmaceutically acceptable salts or optical isomers thereof.

20. A method according to claim 13, wherein the compound of formula II is selected from the group of compounds consisting of 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methylphenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromediylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-[3-chlorophenyl]-4-[pyridin-2-yl]-1,3-oxazole and 2-(2,5,6-trifluorophenyl)-4-(2-pyridyl)-1,3-oxazole, 2-(3-nitrophenyl)-4-(2-pyridyl-1,3-oxazole, 2-(3-bromophenyl)-4-(2-pyridyl)-1,3-oxazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 2-[3-bromophenyl]-4-[pyridin-2-yl]-1,3-oxazole, 2-(3-cyanophenyl)-4-(pyridin-2-yl)-1,3-oxazole, 5-[3-hydroxyphenyl]-3-[pyridine-2-yl]-1,2-oxazole, 5-[3-cyanophenyl]-3-[pyridin-2-yl]-1,2-oxazole, 3-(2-pyridyl)-5-(3-iodophenyl)-1,2,4-triazole, 3-(2-pyridyl)-5-(5-chloro-2-aminophenyl)-1,2,4-oxadiazole and pharmaceutically acceptable salts or optical isomers thereof.

21. A method according to claim 13, wherein the compound of formula III is selected from the group of compounds consisting of 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyl)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazole, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole, 3-(2-pyridyl)-5-(3-bromophenyl)-1,2,4-oxadiazole, 2-(3,5-dichlorophenyl)-4-(2-pyridyl)-1,3-oxazole, 2-(3-chlorophenyl)-4-(2-pyridyl)-1,3-oxazole, 2-(3-methoxyphenyl)-4-(2-pyridyl)-1,3-oxazole, 2-(2-chlorophenyl)-4-(2-pyridyl)-1,3-oxazole, 2-(3-trifluorophenyl)-4-(2-pyridyl)-1,3-oxazole, 2-(3-methyl phenyl)-4-(2-pyridyl)-1,3-oxazole, 2-(1-naphthyl)-4-(2-pyridyl)-1,3-oxazole, 2-(3-trifluoromethoxyphenyl)-4-(2-pyridyl)-1,3-oxazole, 2-(2,3-difluorophenyl)-4-(2-pyridyl)-1,3-oxazole, 2-(2,5-difluorophenyl)-4-(2-pyridyl)-1,3-oxazole, 2-(3,5-difluorophenyl)-4-(2-pyridyl)-1,3-oxazole, 2-(3-cyanophenyl)-4-(2-pyridyl)-1,3-oxazole, 2-(3,5-dimethoxyphenyl)-4-(2-pyridyl)-1,3-oxazole, 2-(2,3-dichlorophenyl)-4-(2-pyridyl)-1,3-oxazole, 2-(3-chloro-5-cyanophenyl)-4-(2-pyridyl)-1,3-oxazole, 2-(3-fluoro-5-cyanophenyl)-4-(2-pyridyl)-1,3-oxazole, 2-(3-chloro-5-fluorophenyl)-4-(2-pyridyl)-1,3-oxazole, 2-(3-cyanophenyl)-4-(5-chloropyrid-2-yl)-1,3-oxazole, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyanophenyl)-4-(3-fluoropyrid-2-yl)-1,3-oxazole, 2-(3,5-dimethoxyphenyl)-4-(5-fluoropyrid-2-yl)-1,3-oxazole, 2-(3-cyanophenyl)-4-(5-methoxypyrid-2-yl)-1,3-oxazole, 2-(3-cyanophenyl)-4-(2-quinolinyl)-1,3-oxazole, 2-(3-cyanophenyl)-4-(3-chloro-5-trifluoromethylpyrid-2-yl)-1,3-oxazole, 2-(5-chloro-2-methoxyphenyl)-4-(2-pyridyl)-1,3-oxazole, 2-(2-chloro-5-methylthiophenyl)-4-(2-pyridyl)-1,3-oxazole, 2-(2-bromo-5-methoxyphenyl)-4-(2-pyridyl)-1,3-oxazole, 2-(2,5,6-trifluorophenyl)-4-(2-pyridyl)-1,3-oxazole, 2-[3-chlorophenyl]-4-[pyridin-2-yl]-1,3-oxazole, 2-(2,5,6-trifluorophenyl)-4-(2-pyridyl)-1,3-oxazole, 2-(3-nitrophenyl)-4-(2-pyridyl)-1,3-oxazole, 2-(3-bromophenyl)-4-(2-pyridyl)-1,3-oxazole and pharmaceutically acceptable salts or optical isomers thereof.

22. A method according to claim 13, wherein the compound of formula II is selected from the group of compounds consisting of 3-(5-Methyl-pyrid-2-yl)-5-(3-cyanophenyl)-1,2,4-oxadiazole, 3-(5-Cyano-pyrid-2-yl)-5-(3-cyanophenyl)-1,2,4-oxadiazole, 3-(2-Pyridyl)-5-(5-bromo-2-methoxyphenyl)-1,2,4-oxadiazole, 3-(2-Pyridyl)-5-(5-bromo-2-fluorophenyl)-1,2,4-oxadiazole, 3-(2-Pyridyl)-5-(5-cyano-2-fluorophenyl)-1,2,4-oxadiazole, 3-(2-Pyridyl)-5-(5-bromopyrid-3-yl)-1,2,4-oxadiazole, 3-(2-Pyridyl)-5-(5-chloro-pyrid-3-yl)-1,2,4-oxadiazole, 3-(5-Cyanopyrid-2-yl)-5-(5-bromo-pyrid-3-yl)-1,2,4-oxadiazole, 3-(5-Fluoropyrid-2-yl)-5-(5-bromo-pyrid-3-yl)-1,2,4-oxadiazole, 3-(2-Pyridyl)-5-(2-thiomethoxy-pyrid-3-yl)-1,2,4-oxadiazole, 3-(2-Pyridyl)-5-(5-methylpyrid-3-yl)-1,2,4-oxadiazole, 3-(2-Pyridyl)-5-(5-methoxypyrid-3-yl)-1,2,4-oxadiazole, 3-(2-Pyridyl)-5-(3-cyano-5-methylphenyl)-1,2,4-oxadiazole, 3-(2-Pyridyl)-5-(3-fluoro-5-bromophenyl)-1,2,4-oxadiazole, 3-(2-Pyridyl)-5-(3-iodo-5-bromophenyl)-1,2,4-oxadiazole, 3-(5-Fluoro-2-pyridyl)-5-(3-fluoro-5-bromophenyl)-1,2,4-oxadiazole, 3-(2-Pyridyl)-5-(3-iodo-5-(methylphenylester)-1,2,4-oxadiazole, 3-(2-Pyridyl)-5-(3-methoxy-5-(methoxycarbonyl)phenyl)-1,2,4-oxadiazole, 3-(2-Pyridyl)-5-(3-bromo-5-cyanophenyl)-1,2,4-oxadiazole, 3-(2-Pyridyl)-5-(5-cyano-3-iodophenyl)-1,2,4-oxadiazole, 3-(5-Cyano-2-pyridyl)-5-(3-bromophenyl)-1,2,4-oxadiazole, 3-(5-Cyano-2-pyridyl)-5-(3-cyano-5-fluorophenyl)-1,2,4-oxadiazole, 3-(5-Cyano-2-pyridyl)-5-(3-bromo-5-fluorophenyl)-1,2,4-oxadiazole, 3-(2-Pyridyl)-5-(5-cyano-2-methoxyphenyl)-1,2,4-oxadiazole, 3-(2-Pyridyl)-5-(2-cyano-5-methoxyphenyl)-1,2,4-oxadiazole, 3-(2-Pyridyl)-5-(5-cyano-pyrid-3-yl)-1,2,4-oxadiazole, 3-(2-Pyridyl)-5-(3-cyano-5-(methoxycarbonyl)phenyl)-1,2,4-oxadiazole, 3-(2-Pyridyl)-5-(5-phenyl-pyrid-3-yl)-1,2,4-oxadiazole, 3-(2-Pyridyl)-5-(3-cyano-5-methoxyphenyl)-1,2,4-oxadiazole, 3-(2-Pyridyl)-5-(3-cyano-5-hydroxyphenyl)-1,2,4-oxadiazole, 3-(2-Pyridyl)-5-(3-cyano-5-propoxyphenyl)-1,2,4-oxadiazole, 2-(3-Cyanophenyl)-4-(pyridin-2-yl)-1,3-thiazole, 2-((3-Bromo-5-iodophenyl)-4-pyridin-2-yl)-1,3-oxazole, 2-(2-Pyridyl)-5-(3-iodophenyl)-1,3,4-oxadiazole, 2-(2-Pyridyl)-5-(3-cyanophenyl)-1,3,4-oxadiazole, 2-(2-Pyridyl)-5-(3-cyanophenyl)-1,3,4-triazole, 3-(5-Chloropyrid-2-yl)-5-(3-cyano-5-fluorophenyl)-1,2,4-oxadiazole, 3-(5-Chloropyrid-2-yl)-5-(3-cyano-5-chlorophenyl)-1,2,4-oxadiazole, 3-(5-Chloropyrid-2-yl)-5-(3-chloro-5-fluorophenyl)-1,2,4-oxadiazole, 3-(5-Chloropyrid-2-yl)-5-(3-cyano-5-methoxyphenyl)-1,2,4-oxadiazole, 3-(5-Fluoropyrid-2-yl)-5-(3-cyano-5-chlorophenyl)-1,2,4-oxadiazole, 3-(5-Fluoropyrid-2-yl)-5-(3-fluoro-5-chlorophenyl)-1,2,4-oxadiazole, 3-(5-Fluoropyrid-2-yl)-5-(3-cyano-5-methoxyphenyl)-1,2,4-oxadiazole, 3-(5-Cyanopyrid-2-yl)-5-(3-cyano-5-chlorophenyl)-1,2,4-oxadiazole, 3-(5-Cyanopyrid-2-yl)-5-(3-fluoro-5-chlorophenyl)-1,2,4-oxadiazole, 3-(5-Cyanopyrid-2-yl)-5-(3-cyano-5-methoxyphenyl)-1,2,4-oxadiazole, 3-(5-Fluoropyrid-2-yl)-5-(3,5-di-cyanophenyl)-1,2,4-oxadiazole, 3-(3-(4-Dimethylaminobutoxy)-pyrid-2-yl)-5-(3-cyano-5-fluorophenyl)-1,2,4-oxadiazole, 3-(3-(5-Dimethylaminopentyloxy)-pyrid-2-yl)-5-(3-Cyano-5-fluorophenyl)-1,2,4-oxadiazole, and [3-(3-(6-Dimethylaminohexyloxy)-pyrid-2-yl)-5-(3-cyano-5-fluorophenyl)-1,2,4-oxadiazole and pharmaceutically acceptable salts or optical isomers thereof.

23. A method according to claim 13, wherein the compound of formula II is selected from the group of compounds consisting of 3-(2-Pyridyl)-5-(3-allyloxy-5-(methoxycarbonyl)phenyl)-1,2,4-oxadiazole, 3-(2-Pyridyl)-5-(3-N,N-dimethylaminophenyl)-1,2,4-oxadiazole), 3-(2-Pyridyl)-5-(3-cyano-5-(4-pyridyl)phenyl)-1,2,4-oxadiazole, 3-(2-Pyridyl)-5-[2-methoxy-5-(4-pyridyl)phenyl]-1,2,4-oxadiazole, 3-(2-pyridyl)-5-[2-fluoro-5-(4-pyridyl)phenyl]-1,2,4-oxadiazole, 3-(2-Pyridyl)-5-(3-fluoro-5-(4-pyridyl)phenyl)-1,2,4-oxadiazole, 3-(2-Pyridyl)-5-(3-fluoro-5-(3-pyridyl)phenyl)-1,2,4-oxadiazole, 3-(2-Pyridyl)-5-[2-fluoro-5-(3-pyridyl)phenyl]-1,2,4-oxediazole, 3-(2-Pyridyl)-5-[2-methoxy-5-(3-pyridyl)phenyl]-1,2,4-oxadiazole, 3-(2-Pyridyl)-5-(3-cyano-5-(3-pyridyl)phenyl)-1,2,4-oxadiazole, 3-(5-Fluoro-2-pyridyl)-5-(3-fluoro-5-(3-pyridyl)phenyl)-1,2,4-oxadiazole, 3-(2-Pyridyl)-5-[5-(3-pyridyl-pyrid-3-yl)]-]-1,2,4-oxadiazole, 3-(5-Fluoropyrid-2-yl)]-5-[5-(3-pyridyl-pyrid-3-yl)]-3-1,2,4-oxadiazole, 3-(5-Cyanopyrid-2-yl)-5-(3-(pyrid-3-yl)phenyl)-1,2,4-oxadiazole, to 3-(5-Cyanopyrid-2-yl)-5-(3-fluoro-5-(pyrid-3-yl)phenyl)-1,2,4-oxadiazole, 3-(2-Pyridyl)-5-(3-cyano-5-(2-pyridyl)phenyl)-1,2,4-oxadiazole, 3-(2-Pyridyl)-5-[2-methoxy-5-(2-pyridyl)phenyl]-1,2,4-oxediazole, 3-(2-Pyridyl)-5-[2-fluoro-5-(2-pyridyl)phenyl]-1,2,4-oxadiazole, 3-(2-Pyridyl)-5-[(3-(3-fluorophenyl)-5-fluorophenyl)]-1,2,4-oxadiazole, 3-(2-Pyridyl)-5-(3-cyano-5-(3-thiophene)phenyl)-1,2,4-oxadiazole, 3-(2-Pyridyl)-5-[5-(3-thienyl)-pyrid-3-yl]-1,2,4-oxadiazole, 3-(2-Pyridyl)-5-[5-3-furyl)-pyrid-3-yl]-1,2,4-oxadiazole, 3-(2-Pyridyl)-5-[5-(3-methoxyphenyl)-pyrid-3-yl]-1,2,4-oxadiazole, 3-(2-Pyridyl)-5-(3-cyano-5-(5-pyrimidyl)phenyl)-1,2,4-oxadiazole, 3-(2-Pyridyl)-5-(3-cyano-5-(3-aminophenyl)phenyl)-1,2,4-oxadiazole, 3-(2-Pyridyl)-5-(3-cyano-5-(3-fluorophenyl)phenyl)-1,2,4-oxediazole, 3-(2-Pyridyl)-5-[5-(5-pyrimidyl)-pyrid-3-yl]-1,2,4-oxadiazole, 3-(2-Pyridyl)-5-(3-aminomethyl-5-cyanophenyl)-1,2,4-oxadiazole, 3-(2-Pyridyl)-5-[5[(2-propenyl)-pyrid-3-yl]-1,2,4-oxediazole, 3-(2-Pyridyl)-5-(3-cyano-5-vinylphenyl)-1,2,4-oxadiazole, 3-(2-Pyridyl)-5-(3-cyano-5-(2-hydroxyethyl)phenyl)-1,2,4-oxadiazole, 3-(2-Pyridyl)-5-(3-cyano-5-(2,3-dichloropropoxy)phenyl)-1,2,4-oxadiazole, 3-(2-Pyridyl)-5-(3-allyloxy-5-carboxyphenyl)-1,2,4-oxadiazole, 3-(2-Pyridyl)-5-(3-allyloxy-5-cyanophenyl)-1,2,4-oxadiazole, 3-(2-Pyridyl)-5-(5-cyano-3-[3-hydroxypropyn-1-yl]phenyl)-1,2,4-oxadiazole, 3-(2-Pyridyl)-5-(2-N-methylaminophenyl)-1,2,4-oxadiazole, and 3-(2-Pyridyl)-5-[5-(3-N-benzyl-1,2,5,6, tetrahydropyridine)-pyrid-3-yl]-1,2,4-oxadiazole.

24. A method according to claim 13, wherein the compound of formula II is selected from the group of compounds consisting of 3-(2-pyridyl)-(3-fluorophenyl)-1,2,4-oxadiazole, 3-(2-pyridyl)-5-(2,3-dimethoxyphenyl)-1,2,4-oxadiazole, 3-(2-pyridyl)-5-(3-phenoxyphenyl)-1,2,4-oxadiazole, 3-(2-pyridyl)-5-(3-benzoylphenyl)-1,2,4-oxadiazole, 3-(2-pyridyl)-5-(2-chloro-5-(trifluoromethyl)phenyl)-1,2,4-oxadiazole, 3-(2-pyridyl)-5-(3,4,5-trifluorophenyl)-1,2,4-oxadiazole, 3-(3-methoxyphenyl)-5-(2-pyridyl)-1,2,4-oxadiazole, 3-(pyrid-2-yl)-5-(2-hydroxyphenyl)-1,2,4-oxadiazole, 3-(2-pyridyl)-5-(5-chloro-2-hydroxyphenyl)-1,2,4-oxadiazole, 3-(2-pyridyl)-5-(2-aminophenyl)-1,2,4-oxadiazole, 3-(2-pyridyl)-5-(5-chloro-2-aminophenyl)-1,2,4-oxadiazole, 5-[3-hydroxyphenyl]-3-[pyridin-2-yl]-1,2-oxazole, 5-[3-cyanophenyl]-3-[pyridin-2-yl]-1,2-oxazole, 3-(2-pyridyl)-5-(3-chlorophenyl)-1,2,4-triazole, 3-(2-pyridyl)-5-(3-iodophenyl)-1,2,4-triazole, 3-(2-pyridyl)-5-(5-hydroxypyrid-3-yl)-1,2,4-oxadiazole, 3-(5-fluoropyrid-2-yl)-5-(5-fluoro-3-(thiomethyl)phenyl)-1,2,4-oxadiazole, 5-(2-pyridyl)-3-[3-(1H-imidazol-1-yl)-5-fluorophenyl)]-1,2,4-oxadiazole, 3-(2-pyridyl)-5-(3-cyano-5-trifluoromethylphenyl)-1,2,4-oxadiazole, 3-(5-fluoro-2-pyridyl)-5-(3-fluoro-5-trifluoromethylphenyl)-1,2,4-oxadiazole, 3-(5-cyanopyrid-2-yl)-5-(3-alloxy-5-cyanophenyl)-1,2,4-oxadiazole, 3-(5-fluoropyrid-2-yl)-5-(3-alloxy-5-cyanophenyl)-1,2,4-oxadiazole, 3-(5-cyanopyrid-2-yl)-5-(3-cyano-5-propoxyphenyl)-1,2,4-oxadiazole, 3-(5-fluoropyrid-2-yl)-5-(3-cyano-5-propoxyphenyl)-1,2,4-oxadiazole, 3-(2-pyridyl)-5-(3-cyano-5-nitrophenyl)-1,2,4-oxadiazole, 3-(5-fluoropyrid-2-yl)-5-(3-cyano-5-nitrophenyl)-1,2,4-oxadiazole, 3-(5-fluoropyrid-2-yl)-5-(3-cyano-5-dimethylaminophenyl)-1,2,4-oxadiazole, 3-(5-fluoropyrid-2-yl)-5-(3-cyano-5-(2-methoxyethoxy)phenyl)-1,2,4-oxadiazole, 3-(5-fluoropyrid-2-yl)-5-(3-cyano-5-(1H-imidazol-1-ylmethyl)phenyl)-1,2,4-oxadiazole, 3-(2-pyridyl)-5-(3-cyano-5-(methoxymethyl)phenyl)-1,2,4-oxadiazole, 3-(3-cyano-5-methoxyphenyl)-5-(2-pyridyl)-1,2,4-oxadiazole, 3-(3-cyano-5-methoxyphenyl)-5-(5-fluoropyrid-2-yl)-1,2,4-oxadiazole, 3-(5-fluoropyrid-2-yl)-5-(3-cyano-5-ethoxyphenyl)-1,2,4-oxadiazole, 3-(5-cyanopyrid-2-yl)-5-(3-cyano-5-ethoxyphenyl)-1,2,4-oxadiazole, 3-(5-chloropyrid-2-yl)-5-(3-allyloxy-5-cyanophenyl)-1,2,4-oxadiazole, 3-(5-chloropyrid-2-yl)-5-(3-cyano-5-propoxyphenyl)-1,2,4-oxadiazole, 3-(5-chloropyrid-2-yl)-5-(3-cyano-5-ethoxyphenyl)-1,2,4-oxadiazole, 3-(5-fluoropyrid-2-yl)-5-(3-cyano-5-hexyloxyphenyl)-1,2,4-oxadiazole, 3-(5-fluoropyrid-2-yl)-5-(3-cyano-5-(methoxymethyl)phenyl)-1,2,4-oxadiazole, 3-(5-fluoropyrid-2-yl)-5-(5-cyano-2-methoxyphenyl)-1,2,4-oxadiazole, 5-(5-cyano-2-methoxyphenyl)-3-(5-cyano-pyrid-2-yl)-1,2,4-oxadiazole, 3-(5-fluoro-pyrid-2-yl)-5-(3-bromophenyl)-1,2,4-oxadiazole, 5-(3-chloro-5-methyl-pyrid-4-yl)-3-(2-pyridyl)-1,2,4-oxadiazole, 5-(3-chloro-5-methoxy-pyrid-4-yl)-3-(2-pyridyl)-1,2,4-oxadiazole, 3-(3-cyano-5-methylphenyl)-5-(2-pyridyl)-1,2,4-oxadiazole, 3-(5-bromo-pyrid-3-yl)-5-(2-pyridyl)-1,2,4-oxadiazole, 3-(3-cyano-5-fluorophenyl)-5-(2-pyridyl)-1,2,4-oxadiazole, 3-(3-iodophenyl)-5-(2-pyridyl)-1,2,4-oxadiazole, 3-(3-cyanophenyl)-5-(2-pyridyl)-1,2,4-oxadiazole, 3-(3-cyano-5-dimethylamino-phenyl)-5-(2-pyridyl)-1,2,4-oxadiazole, 3-(3-cyano-5-methylphenyl)-5-(5-fluoro-pyrid-2-yl)-1,2,4-oxadiazole, 3-(3-cyano-5-fluorophenyl)-5-(5-fluoro-pyrid-2-yl)-1,2,4-oxadiazole, 3-(4-cyanophenyl)-3-(6-cyano-pyrid-2-yl)-1,2,4-oxadiazole, 5-(3-cyano-5-trifluoromethoxyphenyl)-3-(2-pyridyl)-1,2,4-oxadiazole, 5-(3-methoxycarbonyl-5-trifluoromethoxyphenyl)-3-(2-pyridyl)-1,2,4-oxadiazole, 5-(3-cyano-5-trifluoromethoxyphenyl)-3-(5-fluoro-pyrid-2-yl)-1,2,4-oxadiazole, 3-(5-cyano-pyrid-2-yl)-5-(3-cyano-5-trifluoromethoxyphenyl)-1,2,4-oxadiazole, 3-(3-cyano-5-dimethylaminophenyl)-5-(5-fluoro-pyrid-2-yl)-1,2,4-oxadiazole, 5-(5-chloro-pyrid-2-yl)-3-(3-cyano-5-dimethylaminophenyl)-1,2,4-oxadiazole, 5-(5-chloro-pyrid-2-yl)-3-(3-cyano-5-methoxyphenyl)-1,2,4-oxadiazole, 5-(5-chloro-pyrid-2-yl)-3-(6-cyano-4-methoxy-pyrid-2-yl)-1,2,4-oxadiazole, 5-(5-chloro-pyrid-2-yl)-3-(6-cyano-4-hydroxy-pyrid-2-yl)-1,2,4-oxadiazole, 5-(5-chloro-pyrid-2-yl)-3-(3-cyano-5-trifluoromethoxyphenyl)-1,2,4-oxadiazole, 5-(5-chloro-pyrid-2-yl)-3-(3-cyanophenyl)-1,2,4-oxadiazole, 5-(5-chloro-pyrid-2-yl)-3-(3-cyano-5-methylphenyl)-1,2,4-oxadiazole, 5-(5-chloro-pyrid-2-yl)-3-(3-cyano-5-fluorophenyl)-1,2,4-oxadiazole, 3-(3-cyano-5-trifluoromethoxyphenyl)-5-(2-pyridyl)-1,2,4-oxadiazole, 3-(3-fluoro-5-methoxyphenyl)-5-(2-pyridyl)-1,2,4-oxadiazole, 3-(3-cyanophenyl)-5-(5-fluoro-pyrid-2-yl)-1,2,4-oxadiazole, 3-(3-cyano-5-trifluoromethoxyphenyl)-5-(5-fluoro-pyrid-2-yl)-1,2,4-oxadiazole, 3-(3-fluoro-5-methoxyphenyl)-5-(5-fluoropyrid-2-yl)-1,2,4-oxadiazole, 3-(3,5-dimethoxyphenyl)-5-(5-fluoropyrid-2-yl)-1,2,4-oxadiazole, 3-[3-fluoro-5-(1H-imidazol-1-yl)phenyl]-5-(2-pyridyl)-1,2,4-oxadiazole, 3-(5-fluoro-2-pyridyl)-5-(3-fluoro-5-(3-pyridyl)phenyl)-1,2,4-oxadiazole, 3-(5-fluoro-2-pyridyl)-5-(3-bromo-5-(3-pyridyl)phenyl)-1,2,4-oxadiazole, 3-(5-fluoro-2-pyridyl)-5-(3-fluoro-5-methoxyphenyl)-1,2,4-oxadiazole, 3-(pyrid-2-yl)-5-(3-cyano-5-thiomethylphenyl)-1,2,4-oxadiazole, 3-(5-fluoro-pyrid-2-yl)-5-(3-cyano-5-thiomethylphenyl)-1,2,4-oxadiazole, 3-(pyrid-2-yl)-5-(3-fluoro-5-thiomethylphenyl)-1,2,4-oxadiazole, 3-(pyrid-2-yl)-5-(3-fluoro-5-thiomethylsulphoxidephenyl)-1,2,4-oxadiazole, 3-(pyrid-2-yl)-5-(3-fluoro-5-thioethylphenyl)-1,2,4-oxadiazole, 3-(pyrid-2-yl)-5-(5-fluoro-3-thiotertbutylphenyl)-1,2,4-oxadiazole, 3-(5-fluoropyrid-2-yl)-5-(3-cyano-5-methylphenyl)-1,2,4-oxadiazole, 3-(5-cyanopyrid-2-yl)-5-(3-cyano-5-thiomethylphenyl)-1,2,4-oxadiazole, 3-(5-fluoropyrid-2-yl)-5-(4-cyano-2-thienyl)-1,2,4-oxadiazole, 3-(2-pyridyl)-5-(3-cyano-5-trifluoromethylphenyl)-1,2,4-oxadiazole, 3-(5-fluoro-2-pyridyl)-5-(3-cyano-5-trifluoromethylphenyl)-1,2,4-oxadiazole, 3-(5-fluoro-pyrid-2-yl)-5-[3-(3-pyridyl)phenyl]-1,2,4-oxadiazole, 5-[3-methyl-5-(3-pyridyl)-pyrid-4-yl]-3-(2-pyridyl)-1,2,4-oxadiazole, 5-[3-methoxy-5-(3-pyridyl)-pyrid-4-yl]-3-(2-pyridyl)-1,2,4-oxadiazole, 5-(2-pyridyl)-3-[5-(3-pyridyl)-pyrid-3-yl]-1,2,4-oxadiazole, 5-(2-pyridyl)-3-[3-(3-pyridyl)-phenyl]-1,2,4-oxadiazole, 5-(5-fluoro-pyrid-2-yl)-3-[3-fluoro-5-(3-pyridyl)-phenyl]-1,2,4-oxadiazole, 3-(2-pyridyl)-5-(3-carboxy-5-methoxyphenyl)-1,2,4-oxadiazole, 3-(2-pyridyl)-5-(3-(carboxamido)-5-methoxyphenyl)-1,2,4-oxadiazole, 3-(2-pyridyl)-5-(3-cyano-5-(2-N,N-dimethylaminoethoxy)phenyl)-1,2,4-oxadiazole, 3-(2-pyridyl)-5-(3-cyano-5-(N,N-dimethylaminopropoxy)phenyl)-1,2,4-oxadiazole, 3-(2-pyridyl)-5-(3-cyano-5-(2-aminoethoxy)phenyl)-1,2,4-oxadiazole, 3-(2-pyridyl)-5-(3-cyano-5-(2-hydroxyethoxy)phenyl)-1,2,4-oxadiazole, 3-(2-pyridyl)-5-(3-cyano-5-isopropoxyphenyl)-1,2,4-oxadiazole, 3-(2-pyridyl)-5-(3-cyano-5-ethoxyphenyl)-1,2,4-oxadiazole, 3-(2-pyridyl)-5-(3-cyano-5-(2,2,2-trifluoroethoxy)phenyl)-1,2,4-oxadiazole, 3-(2-pyridyl)-5-(3-cyano-5-cyclopropylmethoxyphenyl)-1,2,4-oxadiazole, 3-(2-pyridyl)-5-(3-amino-5-cyanophenyl)-1,2,4-oxadiazole, 3-(5-fluoropyrid-2-yl)-5-(3-amino-5-cyanophenyl)-1,2,4-oxadiazole, 3-(2-pyridyl)-5-(3-cyano-5-(trifluoromethylsulfonyloxy)phenyl)-1,2,4-oxadiazole, 3-(2-pyridyl)-5-(3-cyano-5-(2-methoxy-2-oxoethoxy)phenyl)-1,2,4-oxadiazole, 3-(2-pyridyl)-5-(3-cyano-5-(2-tert-butoxy-2-oxoethoxy)phenyl)-1,2,4-oxadiazole, 3-(2-pyridyl)-5-(3-cyano-5-(methoxymethoxy)phenyl)-1,2,4-oxadiazole, 3-(2-pyridyl)-5-(3-cyano-5-(methoxyethoxy)phenyl)-1,2,4-oxadiazole, 3-(5-fluoropyrid-2-yl)-5-(3-cyano-5-cyclopentylaminophenyl)-1,2,4-oxadiazole, 3-(2-pyridyl)-5-(3-cyano-5-hexyloxyphenyl)-1,2,4-oxadiazole, 3-(2-pyridyl)-5-(3-cyano-5-(dimethylamino)carbonylphenyl)-1,2,4-oxadiazole, 3-(5-fluoropyrid-2-yl)-5-(3-cyano-5-ethylaminophenyl)-1,2,4-oxadiazole, 3-(5-fluoropyrid-2-yl)-5-(3-cyano-5-diethylaminophenyl)-1,2,4-oxadiazole, 3-(5-fluoro-pyrid-2-yl)-5-[3-fluoro-5-(1H-tetraazol-5-yl)-phenyl]-1,2,4-oxadiazole, 5-[3-fluoro-5-(1-methyl-1H-tetraazol-5-yl)-phenyl]-3-(5-fluoro-pyrid-2-yl)-1,2,4-oxadiazole, 3-(5-fluoro-2-pyridyl)-5-(3-(1-benzyl-1,2,5,6-tetrahydropyridin-3-yl)-5-fluorophenyl)-1,2,4-oxadiazole, 3-(5-fluoro-2-pyridyl)-5-(3-fluoro-5-(1H-imidazol-4-yl)phenyl)-1,2,4-oxadiazole, 1-(3-cyanophenyl)-4-(5-fluoro-2-pyridyl)-1H-imidazole, 3-(2-pyridyl)-5-(3-(1H-imidazol-1-yl)-5-thiomethoxyphenyl)-1,2,4-oxadiazole, 3-(3-cyano-5-(1H-imidazol-1-yl)phenyl)-5-(2-pyridyl)-1,2,4-oxadiazole, 3-(2-pyridyl)-5-(3-cyano-5-(1H-imidazol-1-yl)phenyl)-1,2,4-oxadiazole, 2-(3-iodophenyl)-4-(pyridin-2-yl)-1,3-thiazole, 3-(2-pyridyl)-2-(3-cyanophenyl)-furan, 3-(5-fluoro-2-pyridyl)-2-(3-cyanophenyl)-furan, 3-(5-chloro-2-pyridyl)-2-(3-cyanophenyl)-furan, 2-(2-pyridyl)-5-(5-fluoro-3-(1-imidazolyl)phenyl)-furan, 3-(5-(2-pyridyl)-2-furyl)-benzonitrile, 3-(5-(5-chloro-2-pyridyl)-2-furyl)-benzonitrile, 3-(5-(5-cyano-2-pyridyl)-2-furyl)-benzonitrile, 3-(5-(5-fluoro-2-pyridyl)-2-furyl)-benzonitrile, 3-fluoro-5-(5-(2-pyridyl)-2-furyl)-benzonitrile, 2-(3-cyanophenyl)-4-(5-fluoropyrid-2-yl)-1,3-oxazole, 2-(3-cyano-5-fluorophenyl)-4-(5-fluoropyrid-2-yl)-1,3-oxazole, 2-(3-cyano-5-fluorophenyl)-4-(2-pyridyl)-1,3-oxazole, 2-(5-allyloxy-3-cyanophenyl)-4-(5-fluoropyrid-2-yl)-1,3-oxazole, 2-(3-cyano-5-methoxyphenyl)-4-(pyrid-2-yl)-1,3-oxazole, 2-(3-cyano-5-methoxyphenyl)-4-(5-fluoropyrid-2-yl)-1,3-oxazole, 2-(3-cyano-5-n-propyloxyphenyl)-4-(5-fluoropyrid-2-yl)-1,3-oxazole, and 2-(3-cyano-5-methoxyphenyl)-4-(4-pyrid-2-yl)-5-chloro-1,3-oxazole.
